(19) United States
(12) Patent Application Publication
(10) Pub. No.: US 2026/0000905 A1
(43) Pub. Date: Jan. 1, 2026

(54) SYSTEMS AND METHODS FOR DELIVERING PULSED ELECTROMAGNETIC FIELD THERAPY VIA OPERATOR-GUIDED CAPACITIVE COUPLING

(71) Applicant: Havelock David Lloyd, Chandler, AZ (US)

(72) Inventor: Havelock David Lloyd, Chandler, AZ (US)

(21) Appl. No.: 19/253,310

(22) Filed: Jun. 27, 2025

Related U.S. Application Data
(60) Provisional application No. 63/664,919, filed on Jun. 27, 2024.

(51) Int. Cl. A61N 1/40; A61N 1/02; A61N 1/04

(52) U.S. Cl. CPC A61N 1/40; A61N 1/025; A61N 1/0464; A61N 1/0472

(57) ABSTRACT

Systems and techniques may generally be used for delivering pulsed electrostatic fields for therapeutic purposes. In one aspect, an electrostatic-field therapy apparatus may include a power source; a pulse-rate generator that may produce gating pulses; a solid-state switching stage with at least one transistor whose control terminal receives the gating pulses and whose conduction path periodically switches current from the power source; a step-up transformer whose primary winding couples to the switching stage and whose secondary winding may output high-potential pulses; an electrode coupled to the secondary winding; and a dielectric layer disposed over the electrode so that, when a body region is placed adjacent the dielectric layer, the high-potential pulses may be capacitively transferred through the dielectric as a therapeutic electrostatic field.

### BACKGROUND OF THE INVENTION

This application claims priority from U.S. provisional application Ser. No. 63/664,919, filed June 27, which is incorporated by reference.

The present disclosure generally relates to electrotherapy and physiotherapeutic apparatus and more particularly to devices for applying pulsed electrostatic or electromagnetic fields to biological tissue.

### DESCRIPTION OF THE RELATED ART

Existing solutions to the problem being solved often rely on inductive pulsed electromagnetic field therapy (PEMF) coil systems or transcutaneous electrical nerve-stimulation units that employ adhesive electrodes or conductive gels, which may limit patient comfort and require consumable materials.

### SUMMARY OF THE INVENTION

In general, in a first aspect, the technologies described herein relate to an electrostatic-field therapy apparatus that employs a power source, a pulse-rate generator, and a solid-state switching stage to drive a step-up transformer whose secondary winding supplies high-potential pulses to an electrode that is covered by a dielectric layer, the dielectric layer thereby capacitively imparting the pulses as an electrostatic field to a body region positioned adjacent the layer.

In general, in a second aspect, the technologies described herein relate to a method of manufacturing an electrostatic-field therapy apparatus that includes bonding a conductive sheet to a dielectric plate to form a laminated electrode, potting a step-up ignition coil in silicone, mounting a printed-circuit board carrying pulse-generation and transistor-switching or MOSFET-based switching circuitry section within a shielded enclosure compartment, mechanically coupling the coil to the board through a compliant standoff, and electrically interconnecting the board, coil, electrode assembly, and a removable power-supply module according to a wiring schedule.

In general, in a third aspect, the technologies described herein relate to a pulsed electrostatic-therapy system that integrates a pulse-gated electrostatic-field generator with a resistive grounding interface such that, when an operator contacts the grounding interface and touches a living subject positioned near the dielectric-covered electrode, displacement current passes through the practitioner to focus the electrostatic field at a practitioner-selected region of the subject.

Embodiments of the invention may include one or more of the following features. The power source may be a 3- to 14-volt direct-current supply coupled to an energy-storage capacitor bank. The pulse-rate generator may employ dual 555-type timers independently adjustable for frequency and pulse width within approximately 6 Hz to 36 Hz. The electrode may be a planar copper conductor at least 100 cm² in area and 0.5 mm to 1 mm thick. A resistive discharge path of not less than 0.5 MΩ may connect a practitioner-contact surface to earth ground to dissipate displacement current. An enclosure may include thermal-management components such as a heat sink, fan, or temperature sensor that disables the pulse-rate generator upon over-temperature. The dielectric layer may be a polymethyl methacrylate sheet at least 5 mm thick adhesively bonded to the electrode. Manufacturing variants may route a double-insulated cable through a strain-relieved bulkhead, laser-cut the dielectric plate with beveled edges, program micro-controller firmware to limit duty cycle above 20 Hz, and employ a powder-coated aluminum chassis that maintains at least 5 kV creepage and clearance. System embodiments may be housed in a carry-on-sized transport case with foam compartments, provide manual frequency adjustment via touchscreen, mount 600-V-rated transistors on graphite thermal pads inside EMI shielding, store session parameters in non-volatile memory, sweep frequencies using an adaptive control algorithm, operate from a removable 12-V lithium-ion pack, and communicate wirelessly for data transfer and firmware updates.

### BRIEF DESCRIPTION OF THE FIGURES

The appended figures depict certain features of the various aspects described herein and are not to be considered limiting of the scope of this disclosure.

FIG. 1 depicts an ARC Reactor enclosure, according to various examples.

FIG. 2A illustrates a dual-timer pulse-generation circuit that outputs selectable gating pulses according to various examples.

FIG. 2B depicts a MOSFET switching sub-assembly, according to various examples.

FIG. 2C illustrates internal functional blocks of an NE555 timer that provide independent frequency and pulse-width control according to various examples.

FIG. 3 depicts user-facing power-entry and control hardware, according to various examples.

FIG. 4 illustrates a control cluster with a voltmeter and variable resistors for adjusting drive voltage and pulse frequency according to various examples.

FIG. 5A depicts a side-A elevation of a dielectric-encapsulated copper plate electrode according to various examples.

FIG. 5B illustrates a plan view of the copper plate electrode showing its areal extent and dielectric border according to various examples.

FIG. 5C depicts a side-B elevation of the electrode highlighting full edge encapsulation according to various examples.

FIG. 6 illustrates a keyed high-potential connector that couples the enclosure output to the laminated electrode according to various examples.

FIG. 7 depicts an operational flow diagram, according to various examples.

FIG. 8 illustrates a flowchart illustrating a technique for assembling an ARC reactor, according to various examples.

FIG. 9 illustrates a front-panel interface featuring frequency and amplitude knobs, mains inlet, fuse, and power switch according to various examples.

FIG. 10 depicts an example of an ARC reactor in use in a clinical environment, according to various examples.

FIG. 11 a schematic of a pulsed-electromagnetic-field generator circuit, according to various examples.

### DETAILED DESCRIPTION

Systems and techniques described herein may be used to overcome the limitations of traditional methods for applying therapeutic electromagnetic energy to human tissue. Existing coil-based pulsed-electromagnetic-field devices often require bulky inductors, create unwanted eddy currents in surrounding metal furniture, and can induce painful muscle contractions when strong magnetic gradients intersect nerve pathways. Direct-contact electro-stimulation pads reduce some of these drawbacks yet introduce others, such as skin irritation from conductive gels, uneven current distribution across curved anatomy, and consumable costs associated with adhesive electrodes.

To address these issues, the present disclosure provides a capacitive-coupling platform that may generate low-frequency electrostatic pulses without requiring the patient to be electrically wired to the generator. A compact pulse-rate generator may drive a solid-state switching stage, which in turn may energize a step-up transformer connected to a dielectric-covered copper plate situated beneath a treatment surface. A resistive grounding interface may allow a practitioner to stand in a defined return path so that gentle displacement current flows through the practitioner's hands when touching the patient, thereby focusing the field at selected regions while remaining fully non-invasive.

An example technique may include providing a direct-current power module, operating a timer-based pulse-rate generator to create gating pulses, and using those pulses to switch a transistor that intermittently dumps stored energy into a transformer primary. High-potential (high-voltage, yet generally safe) pulses appearing at the secondary may charge an insulated electrode, forming an electrostatic field that capacitively couples through a dielectric layer to tissue positioned nearby.

Consider a rural physical-therapy clinic where electrical service is limited and supply chains for consumable electrode pads are unreliable. A therapist, or operator, may place the dielectric-covered copper plate on a wooden bench, power the portable enclosure from a small solar-charged battery pack, and stand barefoot on the grounding mat. While the patient performs gentle range-of-motion exercises, the therapist lightly traces muscle groups with one hand. Each touch completes a displacement-current loop through the therapist's body, locally concentrating the field at tight fascia bands and enabling real-time adjustment of frequency and amplitude via front-panel knobs. In this context the system may deliver drug-free pain relief and improve circulation without requiring disposables or high-current mains power, supporting community health initiatives in resource-constrained areas.

In the following detailed description, numerous specific details are set forth by way of examples to provide a thorough understanding of the relevant teachings. However, it should be apparent that the present teachings may be practiced without such details. In other instances, well-known structures, functions, methods, procedures, components, and/or circuitry have been described at a relatively high level, without detail, to avoid unnecessarily obscuring aspects of the present teachings.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

The illustrative embodiments are described with respect to certain types of machines. The illustrative embodiments are also described with respect to other scenes, subjects, measurements, devices, data processing systems, environments, components, and applications only as examples. Any specific manifestations of these and other similar artifacts are not intended to be limiting to the disclosure. Any suitable manifestation of these and other similar artifacts can be selected within the scope of the illustrative embodiments.

Furthermore, the illustrative embodiments may be implemented with respect to any type of data, data source, or access to a data source over a data network. Any type of data storage device may provide the data to an embodiment of the disclosure, either locally at a data processing system or over a data network, within the scope of the disclosure. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific surveys, code, hardware, algorithms, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable devices, structures, systems, applications, or architectures therefor, may be used in conjunction with such embodiment of the disclosure within the scope of the disclosure. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Various processes described herein may be implemented by appropriately programmed general purpose computers, special purpose computers, and computing devices. Typically, a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more scripts, or in other forms. The processing may be performed on one or more microprocessors, central processing units (CPUs), computing devices, microcontrollers, digital signal processors, or like devices or any combination thereof. Programs that implement the processing, and the data operated on, may be stored and transmitted using a variety of media. In some cases, hard-wired circuitry or custom hardware may be used in place of, or in combination with, some or all of the software instructions that can implement the processes. Algorithms other than those described may be used.

### Systems and Methods for Delivering Pulsed Electromagnetic Field (PEMF) Therapy Via Therapist-Guided Capacitive Coupling

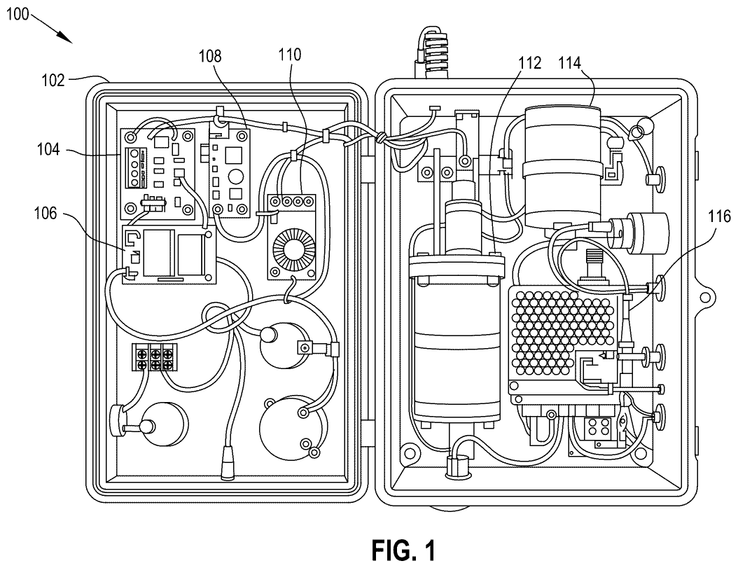

FIG. 1 depicts an ARC Reactor enclosure, according to various examples.

Referring to FIG. 1, in some aspects, an electrostatic-field therapy apparatus may be packaged within an ARC Reactor enclosure 102 that opens clamshell-style to reveal a left electronics bay and a right power-conversion bay. The ARC Reactor enclosure 102 may be molded of glass-fiber-reinforced polycarbonate that provides dielectric strength exceeding 25 kV/mm, and an internal rib network may partition low-voltage logic from high-potential conductors so that at least 5 kV of creepage and clearance distance is maintained between user-accessible surfaces and energized parts. A peripheral tongue-and-groove seal may cooperate with a compression latch (not visible) to attain an IP-54 ingress rating, while internal bosses may accept threaded inserts that support shock-mounted sub-assemblies; these features may enable both hand-carried clinical use and vibration-prone transport scenarios.

In the left bay, a pulse-rate generator 104 may embody a dual-timer architecture that includes two 555-type integrated circuits mounted on a four-layer printed-circuit board. In some implementations, the first timer may operate in an astable configuration to define a selectable repetition rate between about 6 Hz and 36 Hz, and the second timer may operate in a monostable configuration to tailor pulse width independently. The generator 104 may output a gating waveform on a dedicated header that routes through shielded ribbon cable to the gate of a solid-state switching stage; this arrangement may furnish the pulse-rate generator configured to produce gating pulses while also supplying the manually adjustable characteristics.

Adjacent the generator, a Tesla power/input drive FET switch 106 may serve as the transistor-based switching stage. The device may be an N-channel MOSFET rated for at least 600 V drain-to-source and 45 A continuous current, such that its conduction path may periodically divert current from the low-voltage bus toward the primary winding of the step-up transformer described later. A multilayer ceramic gate resistor and a fast-recovery antiparallel diode may be soldered directly across source and drain pads to suppress voltage overshoot, and the MOSFET die may be bonded to an aluminum heat sink that interfaces through graphite phase-change material to the enclosure wall. A negative-temperature-coefficient sensor (not numbered) may be epoxied onto the heat sink and wired back to the pulse-rate generator 104 so that gating pulses may be disabled if device temperature exceeds, for instance, 85° C.

Still in the left bay, a system-voltage adjuster 108 may include a ten-turn wire-wound potentiometer whose wiper feeds the voltage-feedback pin of an internal buck-converter controller inside main VDC supply 116. Rotating the adjuster 108 clockwise may raise converter output from approximately 3 V DC to 14 V DC. A secondary Tesla drive-voltage 110 may reside in series with the adjuster 108 to provide coarse attenuation or user-defined soft-start sequencing; such dual-stage regulation may help practitioners tailor field intensity to sensitive patient populations. The practitioner or practitioners may be, or include, a licensed massage practitioner or therapist. In some embodiments the therapist or practitioner may be a non-human operator, such as a humanoid robot or any automated machine capable of correctly operating the disclosed technologies. Operating may include but is certainly not limited to handling, mobilizing, controlling settings and switching, making informed decisions, and more.

Turning to the right bay, an ignition coil 112 may act as the step-up transformer whose secondary winding provides high-potential output pulses. These pulses are relatively high-voltage for the given application environment, and are generally safe. The coil 112 may originate from an automotive inductive-discharge platform and may possess a primary inductance near 3.2 mH and a turns ratio of roughly 75:1, permitting stepped-up voltages that may exceed 12 kV under open-circuit conditions when excited from a 14 V primary pulse. The coil bobbin may be potted inside silicone elastomer to suppress corona discharge, and compliant standoffs may isolate the coil from vibration transmitted through ARC Reactor enclosure 102.

Immediately beneath the coil, first pulse-storage capacitor 114A and second pulse-storage capacitor 114B may form a parallel energy-storage bank whose aggregate capacitance may approach 470000 μF at 16 V DC. Each capacitor may feature low ESR screw-terminal lugs that are bus-barred with 4 mm copper plate, thereby allowing the bank to discharge rapidly into the primary winding without appreciable internal loss.

Main VDC supply 116 may be an enclosed, synchronous-rectified buck converter capable of accepting 90-264 V AC at a receptacle and producing 3-14 V DC at up to 20 A continuous. The module may incorporate active power-factor correction and a 120° C. thermal cut-out; its chassis ground may bond to the enclosure shell and to the resistive grounding plate described in other figures, thereby forming the discharge path of at least 0.5 MΩ. A detachable harness may allow the supply 116 to be swapped for a lithium-ion battery module.

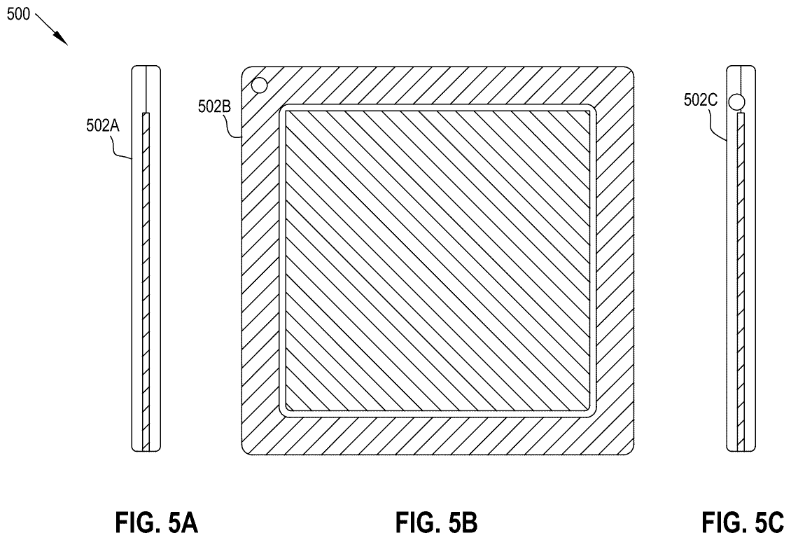

From the primary side of ignition coil 112, heavyweight silicone-insulated leads may pass through an EMI ferrite core before terminating at staked eyelets on the MOSFET drain and positive capacitor rail. On the secondary side, a shielded high-dielectric cable (also known as an HV cable, laser cable, high-voltage cable, or high-potential cable) (not visible in this view) may exit the coil potting cup and route through a bulkhead feed-through toward a laminated copper plate electrode described in FIGS. 5A-5C; this cable may be secured by a connector.

Although the dielectric layer and electrode are not depicted in FIG. 1, the arrangement of components inside ARC Reactor enclosure 102 may position the high-potential feed toward the case hinge so that the electrode cable may deploy naturally when the lid is opened. This internal layout may minimize strain on the secondary lead while keeping the electrode-interface connector remote from the operator controls, reducing inadvertent touch hazard.

A resistive grounding interface (not numbered here) may mate to an exterior jack on ARC Reactor enclosure 102 and may present at least one megohm of resistance between practitioner and earth ground, so that displacement current generated by the electrostatic field may dissipate safely. An internal copper-braid strap may knuckle from that jack to the mains earth pin of an AC input receptacle, further ensuring that any fault current may bypass sensitive electronics.

Finally, wire harnesses inside ARC Reactor enclosure 102 may be laced with PTFE-coated twine and routed through flame-retardant grommets so that conductors carrying high-dV/dt signals remain orthogonal to low-level logic traces, thereby reducing capacitive coupling and radio-frequency emission. Service loops may permit future inclusion of a micro-controller-based adaptive sweep algorithm, while reserved header space on pulse-rate generator 104 may accommodate non-volatile memory for automatic session logging.

Collectively, the elements identified in FIG. 1 may cooperate so that gating pulses from generator 104 modulate Tesla power/input drive FET switch 106, which in turn routes energy from main supply 116 and capacitors 114A/114B into ignition coil 112. The resulting high-potential pulses may then energize a remote electrode beneath a dielectric layer, thereby establishing a therapeutically relevant electrostatic field when a practitioner and patient complete the capacitive pathway.

In several aspects, enclosure 102 may be fabricated from 3 mm-thick glass-fiber-reinforced polycarbonate panels that are laser-cut to ±0.05 mm tolerance and subsequently solvent-welded along dovetailed tongue joints, creating a monocoque shell that may withstand drop impacts of at least 1 kJ without fracture. Internal bosses may accept M3-0.5 insert nuts that receive stainless-steel machine screws coated with MoS₂ dry lubricant, enabling repeated module removal while mitigating galvanic corrosion between dissimilar metals. Edge surfaces may be chamfered at 30 degrees to remove sharp corners, and exterior faces may be lightly bead-blasted to a 0.8 μm Ra finish before receiving an antistatic clear coat that may reduce triboelectric charge build-up during transport.

In other aspects, ignition coil 112 may seat within an elastomeric cradle formed of shore-A 50 silicone that exhibits a glass-transition temperature below −40° C., allowing the cradle to damp vibration across −20° C. to +60° C. ambient ranges commonly encountered in field-use scenarios. The cradle may index via two dowel pins into mating bores on an internal aluminum heat-spreader plate bonded to the lower shell of enclosure 102 using thermally conductive epoxy having a bulk thermal conductivity greater than 3 W m⁻¹ K⁻¹. This plate may also carry the MOSFET heat sink so that waste heat generated in switch 106 and eddy-current losses in coil 112 may be jointly dissipated through the enclosure floor, keeping internal air temperature below 50° C. at a continuous 36 Hz duty cycle.

In many aspects, pulse-storage capacitors 114A and 114B may be secured by stainless-steel clamping brackets lined with PTFE film that accommodates thermal expansion of up to 300 ppm° C.⁻¹ without exceeding a 150 N radial compressive preload that could distort the capacitor sleeve. Torque-controlled M4 fasteners may preload each bracket to 1.2 Nm, and Nylock inserts may prevent loosening under 0-250 Hz vibratory excitation. The bus-bar connection between capacitor terminals and MOSFET drain may consist of a 2 mm-thick C110 copper strap nickel-plated to 5 μm thickness to resist oxidation; the strap may be bent in an “S” profile that provides 4 mm of axial compliance so that solder joint stress is relieved under thermal cycling.

In certain aspects, the printed-circuit board of pulse-rate generator 104 may be suspended on four silicone grommets providing ±1 mm radial float, thereby isolating sensitive analog traces from high-frequency vibration transmitted through enclosure 102. Board-to-board mezzanine connectors rated at 5 A per pin may distribute low-voltage power and gating signals, permitting rapid swap-out of alternate generator cards that, for instance, may implement micro-controller-based adaptive frequency sweeps or Bluetooth telemetry. Header silk-screen may include polarity legends so that service technicians may avoid misconnections, and locating pins may polarize each connector pair.

In several embodiments, main VDC supply 116 may be field-replaceable. A rear battery bay may accept a 12 V, 6 Ah lithium-ion pack whose enclosure may include a keyed blind-mate DC plug and a magnetic latch that generates an audible click when fully seated. An internal shunt switch may disconnect pack output if pack temperature exceeds 60° C., providing an additional layer of thermal protection beyond the MOSFET sensor previously noted. Pack state-of-charge may be reported over an I²C bus to generator 104, enabling the firmware to impose an automatic frequency derate when remaining capacity falls below 20%, thereby extending runtime in home-use settings.

In some aspects, AC input receptacle 306 may mount to the enclosure wall via an IP-67 panel gasket compressed to 20% nominal thickness, yielding a water-resistant seal that may survive disinfectant wipe-down in clinical environments. Terminations may use crimp-style insulated quick-disconnects with pull-strength greater than 50 N, and conductors may be routed through nylon vortex strain-relief bushings so that a 200 N pull on the mains cord may not compromise internal wiring. Fuse 908 may be a ceramic-body, time-delay type tested to IEC 60127-2, minimizing nuisance trips during inrush yet clearing within 10 ms at 250 A fault current.

In other embodiments, ARC reactor enclosure 102 may be produced as a two-piece aluminum housing CNC-milled from 6061-T6 billet and hard-anodized to 25 μm thickness. Such a variant may increase structural rigidity by 70% and may improve EMI containment by 25 dBμV at 200 MHz, desirable in laboratory environments containing sensitive diagnostic instrumentation. The metal chassis may necessitate additional dielectric standoffs for the high-potential routing, yet may allow elimination of an internal EMI shield, reducing part count by two pieces.

In further aspects, pulse rate generator 104 may incorporate a digitally controlled potentiometer that permits front-panel frequency knob 902 to command set-points via a Hall-effect sensor rather than a mechanical shaft, eliminating wear-related drift. Encoder detents may correspond to 0.5 Hz increments, and firmware may average successive readings over 100 ms to suppress jitter. In some examples, a capacitive-sense touch screen may display both instantaneous frequency and peak-to-peak plate voltage, enabling closed-loop adjustment based on patient feedback.

In some embodiments, the grounding interface jack may include a spring-biased switch that opens when the mat plug is removed, disabling MOSFET gate drive and preventing high-potential generation in the absence of a safe return path; this interlock may align with IEC 60601 requirements for Type BF applied parts. A redundant optical coupler may monitor enclosure chassis voltage relative to earth ground, and if more than 50 V RMS is detected the pulse-rate generator may halt within 10 μs, providing rapid fault isolation.

In several aspects, the enclosure hinge may use stainless-steel piano-hinge stock with 2.5 mm pin diameter, coated with PTFE dry film to reduce opening torque below 0.3 Nm. An internal braided copper bonding strap may bridge the hinge leaves, ensuring that the lid remains at chassis potential at all times. Detent angles of 0°, 90°, and 135° may be provided by an integral friction washer, allowing the lid to remain open without additional support in a variety of clinical layouts.

In optional configurations, capacitors 114A/114B may be replaced by an array of twelve 47000 μF snap-in electrolytics arranged in a radial cluster on a fiberglass bus disk. This distributed topology may reduce equivalent series inductance by 40 nH, thereby sharpening pulse edges and improving energy transfer efficiency at higher repetition rates. The cluster may be potted with thermally conductive silicone to create a self-supporting module that can be swapped into legacy enclosures without tooling modifications.

In many aspects, calibration may be performed at the factory by connecting a Rogowski coil around the transformer secondary and measuring pulse-induced flux density. Firmware may then write a gain coefficient to an on-board EEPROM so that the VDC monitor 312 may subsequently display estimated plate field strength in volts-per-meter. Field technicians may re-calibrate by invoking a service menu accessed via a triple-press sequence on system-start switch 310, prompting the firmware to cycle through a predefined test routine.

In some embodiments, a system-start switch may be replaced by a capacitive palm sensor that detects operator presence and automatically arms gate drive when the practitioner places a hand near the control panel, reducing the need for manual actuation in sterile settings. A software timer may disarm the system after 60 s of inactivity, conserving power and mitigating inadvertent operation.

In certain aspects, enclosure 102 may incorporate shock-absorbing corner bumpers molded from thermoplastic polyurethane of shore-A 85 hardness that may deflect up to 6 mm under a 500 N static load, lowering g-forces transmitted to coil 112 during accidental drops. Each bumper may snap into dovetail recesses machined into the enclosure corners, allowing tool-free replacement when worn.

In alternative embodiments, ignition coil 112 may be substituted by a ferrite-core flyback transformer capable of producing similar output amplitudes at lower primary current. A primary-side snubber network comprising 100Ω resistor and 1.5 nF polypropylene capacitor may clamp voltage overshoot, and the flyback winding may be center-tapped to permit bipolar output waveforms that, in some applications, may improve patient comfort by balancing charge displacement.

For safety redundancy, a Hall-effect current sensor may monitor primary-side current and report anomalies to generator 104. If measured current deviates more than 20% from a stored calibration curve, firmware may trigger a pulse-inhibit flag and illuminate a red fault LED adjacent to switch 308, prompting the practitioner to inspect wiring before resuming therapy.

Industrial applicability may span sports-medicine clinics, veterinary rehabilitation, and even aerospace ergonomics laboratories where non-contact stimulation of deep-tissue structures may expedite crew recovery after high-g maneuvers. The modular architecture disclosed herein may allow manufacturers to license individual sub-assemblies—such as the battery bay or adaptive frequency board—to third-party OEMs seeking to integrate pulsed electrostatic therapies into massage tables, wearable braces, or robotic exoskeletons.

In some embodiments, the input drive FET switch 106 may be paralleled by a second identical device with source-sense resistors that equalize current sharing, thereby enabling higher average power for large-area electrode arrays. A Kelvin-connected gate resistor may minimize ringing, and the devices may be mounted on opposite sides of the heat-spreader to distribute thermal load evenly.

Preferably, high-potential cable exiting coil 112 may be silicone-jacketed, rated at 40 kV RMS, and terminated with crimped beryllium-copper contacts that mate with connector 602 using a bayonet lock that requires 30° rotation and 45 N insertion force. In rain-forest deployments, the cable jacket may additionally receive a parylene-C conformal coating to resist fungal growth.

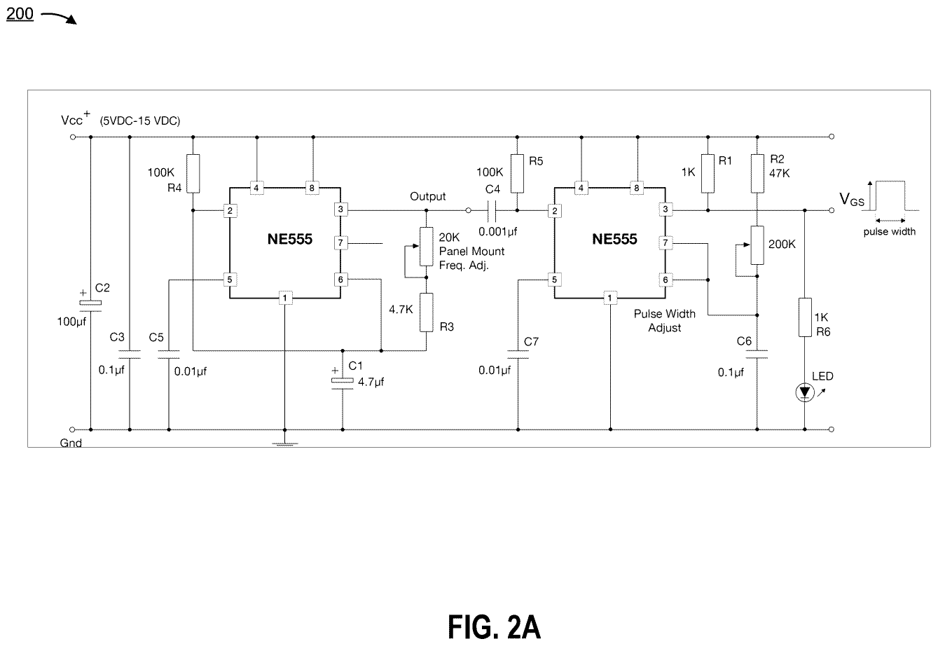

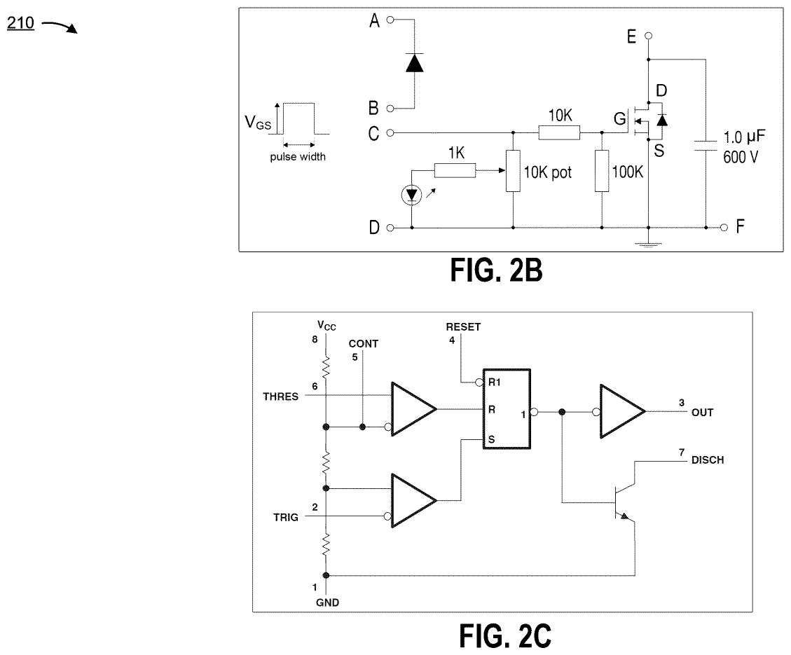

FIG. 2A illustrates a dual-timer pulse-generation circuit that outputs selectable gating pulses according to various examples.

In some aspects, FIG. 2A illustrates a pulse-rate generator 104 that may employ two NE555-type timer integrated circuits arranged to yield independently selectable frequency and pulse-width characteristics suitable for driving the gate of a transistor-based switching stage. A first timer, shown at the left side of the schematic, may operate in an astable configuration in which timing capacitor C4 and timing resistor R4 cooperate with a front-panel frequency-adjust potentiometer to define a repetition rate that may be varied between roughly 6 Hz and 36 Hz. The capacitor C4 may have a nominal capacitance near 0.001 μF, while resistor R4 may exhibit a resistance of about 100 kΩ; the potentiometer may span 20 kΩ so that, when its wiper is rotated, the RC time constant and therefore the frequency may change in a quasi-linear fashion. A node labeled “Output” may be positioned at the junction of the first timer's OUT pin and the leading edge of coupling network R5, allowing technicians to connect an oscilloscope probe and verify compliance with duty-cycle limits that, in some embodiments, may be restricted by firmware to less than thirty percent when frequencies exceed 20 Hz.

A supply rail, denoted VCC, may furnish between 5 V and 15 V to both timers. That rail may be derived from a low-noise linear regulator placed downstream of the main power source and may be decoupled locally by electrolytic capacitor C2 (approximately 100 μF) in parallel with ceramic capacitor C3 (approximately 0.1 μF). These capacitors may suppress line-borne ripple and mitigate load-induced transients, thereby ensuring that comparator thresholds internal to each timer remain stable as frequency is swept. In certain aspects, the same rail may also power an on-board micro-controller that, in future implementations, may implement an adaptive sweep algorithm.

The right-hand timer may function in a monostable topology so that each low-going transition from the first timer triggers a single, width-defined pulse. Its timing network may include fixed resistor R1 (1 kΩ), adjustable resistor R2 (47 kΩ), a pulse-width-adjust potentiometer (200 kΩ), and timing capacitor C6 (0.1 μF). By moving the wiper of a potentiometer, an operator may vary the on-time from roughly 2 ms to 20 ms, an interval that directly modulates energy delivered to the primary winding of a step-up transformer described elsewhere. A bypass capacitor C7 (0.01 μF) may be connected between the monostable timer's control-voltage pin and ground so that noise on the 2/3 VCC reference point is minimized, which may in turn reduce jitter in VGS timing.

The output pin of the second timer may route through current-limit resistor R6 (1 kΩ) to an indicator LED that flashes in concert with each generated pulse. The same node may feed gate-shaping resistor R3 (4.7 kΩ) located on a separate driver board, thereby delivering a square-edged gating waveform to the MOSFET gate within Tesla power/input drive FET switch 106. In some embodiments, a steering diode (not separately numbered in this schematic) may clamp the gate negative-going excursion to approximately −0.6 V, thus protecting the transistor control terminal against reverse bias conditions that could arise when the step-up transformer discharges.

A coupling capacitor C5 (0.01 μF) may straddle the VCC rail and the output node of the first timer so that rise-time steepness is limited in a controlled manner, reducing electromagnetic interference that might otherwise propagate along conductors linking the pulse-rate generator 104 to wiring harnesses inside ARC Reactor enclosure 102. Such EMI mitigation may be valuable when the apparatus is deployed in electromagnetically sensitive clinical environments or near diagnostic imaging equipment.

Ground reference for every component in FIG. 2A may terminate at a single copper flood beneath the dual-timer PCB, which may be bonded to the main chassis ground by a low-inductance braid. This star-ground philosophy may ensure that displacement current does not couple into low-level logic even while multi-kilovolt electrostatic pulses are being generated elsewhere in the system.

In many aspects, one or more potentiometers may employ shafts fitted with locking bushings so that once a practitioner establishes a preferred protocol they may tighten a set screw and prevent accidental rotation. The knob attached to one potentiometer may be labeled in 2 Hz divisions and may include a tactile detent at the Schumann-resonance-adjacent value of 7.8 Hz, whereas the knob associated with another potentiometer may be labeled in milliseconds and may feature a finer pitch so that small changes in pulse width may be performed with repeatable precision.

To simplify calibration during manufacture, node “Output” may be factory-populated with a right-angle test post so that automated test equipment may clip on without manual intervention. During such calibration, technicians may measure both repetition rate and duty cycle, then may record those parameters into non-volatile memory, thereby enabling downstream retrieval of build data for service or regulatory purposes.

Although FIG. 2A depicts discrete through-hole resistors and axial electrolytics, in alternative embodiments the entire pulse-rate generator 104 may be implemented with surface-mount components or may be migrated into firmware running on a micro-controller that synthesizes gating pulses via a timer peripheral. Such a micro-controller may permit additional features such as dual-slope ramping of repetition rate, closed-loop duty-cycle enforcement, or Bluetooth telemetry compatible with a wireless module.

When assembled, the dual-timer PCB may slot into a keyed receptacle mounted to an aluminum shield wall within ARC Reactor enclosure 102. That shield wall may subdivide the interior so that sensitive logic remains demonstrably isolated from the high-potential chamber that houses ignition coil 112 and energy-storage capacitors 114A and 114B. A two-pin pluggable header may carry the VGS waveform across that barrier, while a separate three-pin header may transport VCC, ground, and a temperature-fault line that may asynchronously reset both timers should the aforementioned thermal sensor detect over-temperature. Pulling the RESET pin of either timer low forces its output inactive, thereby halting production of gating pulses and safeguarding the Tesla power/input drive FET switch 106 and transformer primary winding.

In some aspects, FIG. 2B illustrates a gate-drive and power-switching sub-assembly that may translate the low-voltage gating waveform originating at the pulse-rate generator into high-current pulses suitable for energising a primary winding of a step-up transformer. A pulsed control signal, labelled “VGS” in the figure and appearing at node B, may enter through an arrow that designates the square-wave output produced by the dual-timer circuit discussed previously. From node B, the waveform may encounter a steering diode positioned with its anode tied to node B and its cathode tied to intermediate node C; this diode may ensure that only positive-going edges propagate toward the MOSFET gate while negative excursions are clamped at approximately −0.7 V, thereby preventing reverse bias of the transistor's control terminal and reducing cross-conduction risk.

Downstream of the diode, a series resistor identified as 1 K may appear inline between node C and a variable-bias network. The resistor 1 K may limit surge current into the gate during fast transitions and may cooperate with the distributed capacitance of the MOSFET to shape rise-time to a value that balances switching speed against radiated electromagnetic interference. Node C may also provide a convenient oscilloscope test point so that service personnel may verify gate-drive amplitude in situ without disturbing the remainder of the circuit.

The adjustable network may include a linear potentiometer marked 10K pot wired as a voltage divider between node Cand common return node D. By rotating the wiper of the 10K pot, an operator may set the peak VGS amplitude in a range that may span, for example, 6 V to 12 V, thus tailoring the MOSFET's conduction interval to match the magnitude of the low-voltage bus set by the system-voltage adjuster. In parallel with the potentiometer, a fixed resistor labelled 100K may connect from the MOSFET gate G to source node F, ensuring that the gate is positively referenced to ground whenever the incoming pulses are absent, thereby safeguarding against unintended turn-on during power-up or fault conditions.

A 10 kΩ pull-up resistor, indicated simply as 10K, may join node C to node E so that the gate sees full pulse amplitude when the 10K pot is rotated fully clockwise; this resistor 10K may also define an upper-bound impedance that, in conjunction with gate capacitance, establishes an RC constant which may limit dV/dt to values that comply with electromagnetic-compatibility guidelines in hospital environments.

The heart of the switching stage may be an N-channel enhancement-mode MOSFET whose gate is labelled G, drain D, and source S (coincident with node F). The MOSFET may exhibit a drain-to-source avalanche rating greater than 600 V and a continuous current rating on the order of 30 A. The drain D may connect to node E, which in turn routes to one side of the step-up transformer primary winding located on a separate power board; the source S may bond to ground node F, thus completing the conduction path through which current from the power source may be periodically switched.

Across the drain-to-source terminals, a polypropylene snubber capacitor identified as 1.0 μF 600 V may be soldered with minimal lead length. This capacitor 1.0 μF 600 V may absorb voltage spikes produced by leakage inductance of the transformer primary and may cooperate with intrinsic MOSFET capacitances to form an RCD network that limits peak drain stress, for instance to below 450 V when the low-voltage bus is operating at its maximum of 14 V. In certain aspects, the capacitor may exhibit an ESR below 15 mΩ so that it can repeatedly handle 10 ms-width charging events without significant self-heating, thereby supporting the thermal-management objectives.

Reference node A, located above the diode cathode, may be tied via a reverse-polarity blocking network to the high-potential secondary winding and may supply a return path for free-wheel current when the MOSFET turns off. Although not explicitly terminated in FIG. 2B, node A may connect to a clamp circuit located near the ignition coil so that over-voltage events appearing at the secondary do not propagate backward into the logic ground domain.

Ground node D may be common to both the dual-timer pulse-rate generator and the power-switching board. A heavy copper pour under node D may tie into the enclosure's internal ground spine that, in turn, mates to the resistive grounding interface described in other views, thereby maintaining a single-point reference that assists in dissipating displacement current. Node F—the MOSFET source—may also be hard-soldered to that same copper ground plane, ensuring that the gate-to-source voltage is measured with respect to a low-impedance reference, which is particularly beneficial when the apparatus must pass electrical-fast-transient testing.

A mechanical outline surrounding the sub-assembly suggests that all components shown in FIG. 2B may be mounted on a dedicated daughter card that inserts vertically into the ARC Reactor enclosure 102. The board edge may feature gold-plated fingers that engage a high-current backplane connector, delivering low-voltage bus power from the main VDC supply and transporting gate pulses via a differential pair to suppress common-mode noise. Such modularity may facilitate field replacement or future upgrades, for example substituting a SiC FET to improve switching efficiency without altering upstream pulse-generation logic.

During operation, a positive edge arriving at node B may charge the MOSFET gate through resistor 1 K and the 10K pot, raising VGS above the threshold so that the transistor enters its conductive region. Current from energy-storage capacitors 114A and 114B may then flow through the transformer primary, storing magnetic energy that, upon gate closure, is transferred to the secondary and ultimately the therapeutic electrode. When the gating waveform returns low, the diode between nodes B and C may rapidly discharge the gate through the 100K resistor, bringing the MOSFET back to its off state within microseconds and limiting pulse width to the time constant defined in FIG. 2A.

Because both the variable-bias potentiometer 10K pot and fixed resistor 100K are accessible from the board edge, factory technicians may dial in a desired gate amplitude that strikes a balance between switching losses and transformer saturation, thereby allowing the apparatus to accommodate power-supply variations. In an alternative embodiment, the potentiometer may be replaced with a digitally controlled resistor so that a micro-controller could automatically adjust gate drive in response to temperature feedback, thus integrating seamlessly with the over-temperature shut-down feature.

Collectively, the labelled elements in FIG. 2B may form a solid-state switching stage including at least one transistor having a control terminal driven by the gating pulses and a conduction path arranged to periodically switch current from the power source, while the presence of the snubber capacitor 1.0 μF 600 V and gate-bias network comprising 10K, 10K pot, and 100K may furnish optional configurability and protection pathways that support robust, long-term therapeutic operation across a wide range of clinical and portable use-case scenarios.

FIG. 2C illustrates internal functional blocks of an NE555 timer that provide independent frequency and pulse-width control according to various examples.

In some aspects, FIG. 2C illustrates an internal functional diagram of an NE555-type timing integrated circuit that may reside within the pulse-rate generator described earlier and that may provide the foundational sub-blocks required to create selectable gating pulses. A supply pin labelled VCC 8 may accept a regulated rail that, in many embodiments, may lie between 5 V and 15 V so that the timing network operates reliably across the entire 3 V-14 V low-voltage window furnished by the power source. A ground pin designated GND 1 may complete the supply loop and may also serve as the zero-volt reference for threshold comparators inside the device, thereby tying all internal decision nodes to a common potential that is co-planar with the enclosure ground plane.

Internally, three equal-value resistors may form a voltage-divider ladder between VCC 8 and GND 1, establishing two tap points: a first reference at two-thirds of VCC and a second reference at one-third of VCC. These taps may feed a high-side comparator and a low-side comparator, respectively. The non-inverting input of the upper comparator may connect to the divider's two-thirds node while its inverting input may connect to the external threshold pin labelled THRES 6. Conversely, the non-inverting input of the lower comparator may connect to the external trigger pin marked TRIG 2, whereas its inverting input may be tied to the one-third-VCC node. In operation, when a voltage applied to THRES 6 rises above two-thirds VCC, the upper comparator may output a logic-high that may reset an internal R-S latch; when a voltage at TRIG 2 falls below one-third VCC, the lower comparator may set that same latch.

The R-S latch may reside at the center of the diagram and may include an asynchronous clear input driven by an external RESET 4 pin. Pulling RESET 4 low may force the latch into its reset state regardless of comparator outputs, thereby rendering the OUT buffer inactive and simultaneously switching on an internal open-collector transistor tied to DISCH 7. For fault-handling purposes, a temperature sensor or lid-interlock switch located elsewhere in the apparatus may assert RESET 4 so that high-potential generation ceases instantaneously when unsafe conditions are detected, aligning with the safety strategy.

A buffer stage may follow the latch and may drive the output pin OUT 3. When the latch is set, OUT 3 may present a logic-high compatible with transistor gate drive; when the latch is reset, OUT 3 may present a logic-low. Because the buffer in an NE555 may typically sink or source at least 200 mA, OUT 3 may easily charge gate-shaping networks such as those shown in FIG. 2B, yet in embodiments where EMI minimisation is critical, a series resistor may be added externally to slow edge rates.

Concurrent with the state of OUT 3, an internal NPN transistor may couple pin DISCH 7 to GND 1 when the latch is reset. This action may rapidly discharge an external timing capacitor that is placed between THRES 6 and GND 1, thereby preparing the capacitor for the next charge cycle driven through an external timing resistor. When the latch is set and OUT 3 rises, the DISCH 7 transistor may turn off, allowing the timing capacitor to ramp toward VCC 8 through that resistor, an event that ultimately pushes THRES 6 above two-thirds VCC and again resets the latch, thus completing an astable cycle consistent with the selectable 6 Hz-36 Hz range.

A control-voltage pin CONT 5 may break out the midpoint of the internal resistor ladder so that external modulation may shift the comparator thresholds. In some embodiments, a low-impedance node from a function generator may be injected at CONT 5 through a 0.01 μF capacitor so that the effective repetition rate sweeps across a defined frequency band, an optional feature that may support adaptive therapy protocols. Because CONT 5 is sensitive to noise, a 10 nF capacitor is often connected to ground at that pin, as hinted by the small bypass capacitor icon near CONT 5 in FIG. 2C.

All pins drawn toward the left side of the figure may be shown terminating at open circles, indicating external connectivity to the discrete networks depicted in FIG. 2A. Arrows at TRIG 2 and THRES 6 may portray charge and discharge paths for an external capacitor, while the arrow on DISCH 7 may emphasise the collector of the internal transistor pulling toward GND 1 when active. A thicker line emanating from OUT 3 may suggest its role as a high-current driver, leading ultimately to the transistor gate in FIG. 2B. Preferably, the transistor is a Metal Oxide Semiconductor Field Effect Transistor (MOSFET) although it could be other types.

Because FIG. 2C exposes individual functional blocks rather than physical geometries, the drawing may enable a practitioner to substitute equivalent timing devices—such as CMOS-based LMC555 parts—simply by ensuring that the comparator thresholds and discharge-switch behavior remain consistent. Such flexibility may be valuable when designing a battery-operated version, where quiescent current must be minimized.

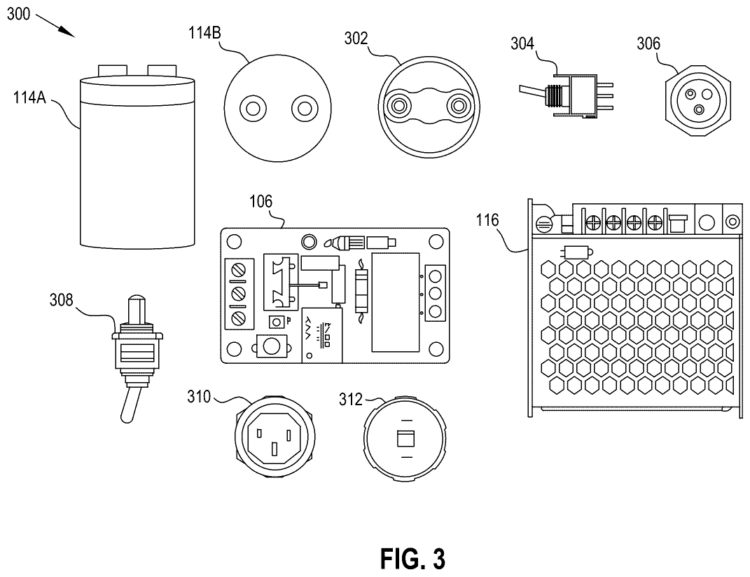

In some aspects, FIG. 3 depicts an operator-facing control cluster that may be mounted on a front or side wall of the ARC Reactor enclosure 102 and that may enable a practitioner to energize, arm, and continuously monitor the electrostatic-field therapy apparatus without exposing high-potential conductors. From left to right, the illustrated panel may first present a Tesla generator 302, which may correspond to the secondary-side bushing of the step-up transformer whose secondary winding provides high-potential output pulses. The bushing of 302 may accept a concentric spring contact that mates to a shielded cable routed toward the laminated electrode; a molded keyway may prevent mis-alignment, and an internal skirt may shroud any corona discharge so that ionization does not propagate toward adjacent plastic surfaces.

Immediately to the right of Tesla generator 302, an AC power switch 304 may be configured as an illuminated, double-pole rocker that, when toggled to its “on” position, may connect both line and neutral conductors from an upstream mains cord to an internal fuse and, subsequently, to the primary of a switching supply that forms part of the power source. The switch body of 304 may integrate a neon indicator driven from the line conductor so that visual confirmation of mains energization remains available even when downstream low-voltage rails have not yet ramped. In certain embodiments, AC power switch 304 may be rated for 250 V AC at 10 A and may include a built-in suppression capacitor that may reduce electromagnetic interference emitted back onto the facility wiring.

Directly adjacent, an AC input receptacle 306 may appear as a recessed, three-pin IEC C14 socket whose earth pin may be bonded to the metallic chassis of ARC Reactor enclosure 102 and, through that chassis, to the resistive grounding interface. The AC input receptacle 306 may incorporate an integral filter network consisting of X-class capacitors and a common-mode choke so that differential and common-mode noise generated during high-potential pulsing is substantially attenuated before it can egress onto the power grid. Installation of AC input receptacle 306 in a folded-metal pocket may ensure that the mating cord set remains strain-relieved when the enclosure is moved or re-oriented.

Centered in the illustrated row, a system-on switch 308 may be embodied as a low-voltage toggle that receives filtered 24 V DC from an auxiliary rail inside the main VDC supply and may distribute that voltage to subsystems such as the pulse-rate generator 104, the system-voltage adjuster 108, and the user-interface electronics that support session logging. Because switch 308 interrupts only low-energy conductors, its actuation may be performed safely even while the enclosure lid is open during servicing. The toggle lever may feature a red safety cover in some configurations so that inadvertent activation is discouraged when maintenance personnel are working near the high-potential circuitry.

To the right of 308, a momentary system-start switch 310 may be implemented as a rugged, vandal-resistant push-button having an LED-backlit bezel that glows amber when preparatory conditions are met—namely, AC power present, low-voltage rails in regulation, lid interlock closed, and coil temperature below a predetermined threshold. Depressing system-start switch 310 may route a logic-high to the gate-enable input of the solid-state Tesla power/input drive FET switch 106, thereby allowing gating pulses generated by the dual-timer network to reach the MOSFET-based gate. Releasing system-start switch 310 may not de-energize the system; instead, a latching relay or micro-controller flag may sustain the run state until either a fault arises or the practitioner toggles switch 308 off, providing an intuitive two-handed arming sequence that aligns with clinical safety norms.

Adjacent to system-start switch 310, a circular system VDC monitor 312 may provide real-time numeric or bar-graph indication of the low-voltage bus furnished by main VDC supply 116. In some embodiments, VDC monitor 312 may employ an LED-backlit, three-digit seven-segment module capable of displaying 3.0 V to 14.0 V with 0.1-volt resolution, thereby allowing a practitioner to confirm that amplitude adjustments performed via downstream potentiometers remain within the safe-operating window. A protective glass cover over VDC monitor 312 may include an anti-reflective coating so that readings remain legible under the bright lighting typical of therapy rooms.

Dashed arrows shown between the various switches and the underlying circuitry may symbolize control-signal flow rather than power flow. One arrow may extend from AC power switch 304 toward AC input receptacle 306, indicating that mains current traverses the switch before reaching the receptacle's filter; another arrow may extend from momentary system-start switch 310 toward Tesla generator 302, suggesting that actuation of system-start switch 310 initiates the gate-drive path ultimately responsible for high-potential pulses appearing at Tesla generator 302. By depicting the functional linkage in this arrow-based manner, FIG. 3 may clarify to assemblers that low-voltage interlock conductors and high-potential pulse conductors follow distinct harness routes.

Surrounding hardware such as bezel screws, label plates, and silicone gaskets may not carry numeric identifiers in FIG. 3, yet they may collectively confer an IP-54 to IP-65 ingress rating depending on selected materials. For example, a compression gasket under the faceplate carrying AC power switch 304, system on switch 308, and system start switch 310 may be molded of 60-shore-A EPDM so that both dust and low-pressure water spray are excluded from internal circuitry, enhancing reliability for mobile practitioners who transport the enclosure in wetter climates.

While FIG. 3 emphasizes discrete electromechanical operators, alternative embodiments may integrate a capacitive-sense touchscreen that replaces switches 308, 310, and VDC monitor 312 with software buttons and graphical readouts. Such a touchscreen may satisfy the manually adjustable and indicating features, provided that the software incorporates debounce logic and watchdog resets equivalent to the hardware interlocks shown. Regardless of interface style, each control element depicted in FIG. 3 operates conditionally to manage power flow from mains to the low-voltage rail, to enable gate drive to the transistor, and to provide immediate operator feedback-thereby supporting safe initiation and continual supervision of the electrostatic-field therapy sequence.

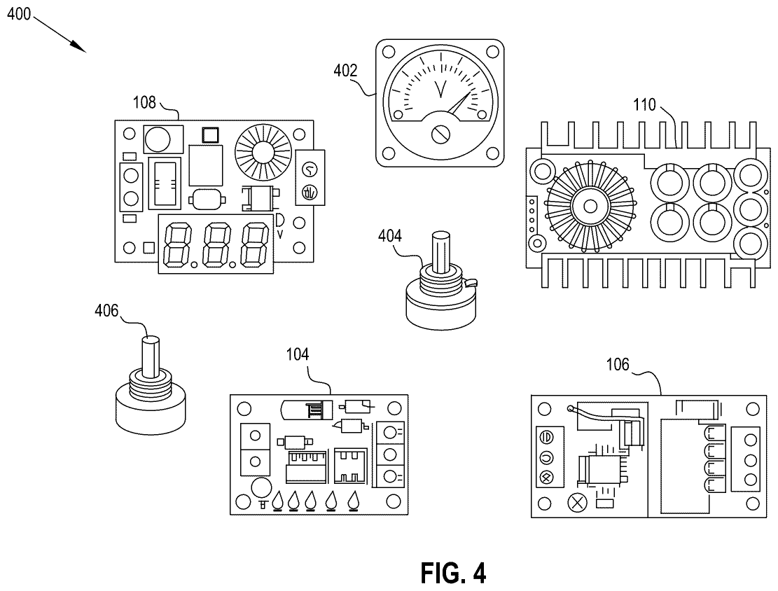

In some aspects, FIG. 4 illustrates a front-panel calibration cluster that may reside on the upper deck of the ARC Reactor enclosure 102 and that may provide tactile, low-voltage access to the two principal therapy-shaping parameters: direct-current supply amplitude and pulse-rate generator repetition rate. Centrally positioned, a VC input readout 402 may take the form of an analog, moving-coil voltmeter whose dial is graduated from 0 V to 15 V, thereby encompassing the full 3 V-to-14 V operating span. A mirrored inner scale and knife-edge pointer may help eliminate parallax so that a practitioner can resolve set-points to within ±0.1 V. In certain embodiments, the meter mechanism may be damped with silicone oil so that the pointer settles quickly even when the underlying buck converter responds to dynamic load changes produced by the Tesla power/input drive FET switch 106.

A pair of insulated ring terminals may connect the rear studs of VC input readout 402 directly across the low-voltage bus emanating from main VDC supply 116. Because the meter draws only micro-ampere-level current, its insertion may negligibly load the supply yet may continuously confirm that adjustments imparted via the adjacent potentiometers remain inside a user-defined therapeutic envelope. A dashed arrow printed on the overlay may run from VC input readout 402 toward variable resistor 404, indicating that the numerical indication of VC input readout 402 corresponds directly to manipulations of that potentiometer.

To the lower left of VC input readout 402, an amplitude-control potentiometer labelled R9 5 kΩ variable resistor 404 may protrude through the panel. The potentiometer shaft may accept a slotted trim screw rather than a large external knob, signaling that this control is intended for infrequent adjustment—such as when tailoring maximum field intensity to a particular patient profile—rather than for continual real-time modulation. A circular lock-nut may retain variable resistor 404 against the interior surface of the enclosure, compressing an O-ring that confers splash resistance and vibration damping during transport.

Internally, one fixed end of variable resistor 404 may tie to the feedback pin of a synchronous buck-converter controller embedded within main VDC supply 116, while the opposing fixed end may bond to a precision resistor network that defines the lower leg of the feedback divider. Rotating the wiper clockwise may decrease the overall divider ratio so that the controller responds by raising the supply output, incrementally increasing the stored energy per pulse that is ultimately delivered to ignition coil 112. Conversely, rotating the wiper counter-clockwise may reduce output voltage, allowing sensitive subjects to receive a gentler electrostatic field. Because the converter may regulate at up to 20 A continuous, the 5 kΩ value of variable resistor 404 may assure that only micro-ampere currents flow through the feedback network, thereby minimizing dissipation and long-term drift.

To the lower right of VC input readout 402, a frequency-control potentiometer labelled R8 20 kΩ variable resistor 406 may mount flush with the front panel and may employ a knurled, finger-friendly knob that protrudes sufficiently to permit easy, incremental adjustments even while the practitioner maintains eye contact with a patient. Pointer markings on the knob skirt may align with a silk-screened arc that enumerates nominal frequency set-points at 6 Hz, 10 Hz, 15 Hz, 20 Hz, 30 Hz, and 36 Hz—a selectable repetition-rate band. An internal detent at approximately 7.8 Hz may correspond to a Schumann-resonance-adjacent frequency that some operators value for relaxation protocols, though the detent may be subtle so that smooth continuous sweep remains possible.

Variable resistor 406 may wire to the first-timer section of pulse-rate generator 104 by way of a shielded two-conductor harness whose braided drain terminates at the enclosure ground plane. The wiper of variable resistor 406 may form one element of the RC timing network, and altering its resistance may adjust the charge-discharge interval of the connected timing capacitor, thereby linearly varying the period of the gating waveform supplied to the MOSFET gate through the circuitry shown in earlier figures. A second dashed arrow on the panel graphic may extend from variable resistor 406 toward VC input readout 402, reminding users that frequency changes may indirectly influence low-voltage rail sag under high-load duty cycles, a relationship they can monitor in real time.

Between variable resistor 404 and 406, the faceplate may carry laser-etched legends reading “Amplitude (Volts)” and “Frequency (Hz),” respectively, to reduce setup errors in busy clinical settings. A translucent polycarbonate overlay may shield the legends from abrasion while permitting back-lighting for low-ambient-light environments. The overlay may also integrate a photoluminescent strip so that the controls remain identifiable during dimmed, relaxation-oriented therapy sessions.

Electrically, both potentiometers may interface to their downstream circuits via locking JST-style connectors located immediately behind the panel, facilitating rapid replacement should either component drift or suffer contamination. Heat-shrink-covered splices may be avoided so that any field-service operation can occur without hot-work tools. Furthermore, lead dress behind the panel may observe a minimum 8 mm separation between low-level frequency conductors and the high-current supply busses feeding pulse-storage capacitors 114A and 114B.

An optional third, un-populated aperture to the right of variable resistor 406 may accept a small rotary encoder in future embodiments that implement digital frequency control linked to a micro-controller sweep algorithm. Even in units where the encoder is absent, the aperture may be sealed with a threaded aluminum plug so that later upgrades can be executed without re-machining the enclosure.

While FIG. 4 concentrates on manual analog controls, certain production variants may replace VC input readout 402 with a graphical OLED module that simultaneously displays voltage, frequency, and cumulative pulse count. In such instances, variable resistor 406 may be repurposed as a rotary-push encoder that sends quadrature signals to an embedded controller, yet the panel layout may remain substantially as shown so that part-commonality across model tiers is maximized.

In some aspects, FIGS. 5A-5C collectively illustrate a laminated electrode assembly that may serve as the capacitive-coupling interface through which high-potential output pulses are delivered to a living subject. A plan view of the assembly appears in FIG. 5B and is designated copper plate 502B; orthogonal side elevations appear in FIG. 5A and FIG. 5C and are labelled 502A and 502C, respectively. The three views cooperate to convey overall footprint, material stack-up, and edge treatment, thereby enabling a practitioner of ordinary skill to fabricate an electrode that satisfies planar-conductor and dielectric-barrier requirements.

In FIG. 5B, the copper plate 502B may be shown as a square sheet whose shaded interior region represents a conductive foil or rolled-annealed plate having a nominal thickness between about 0.5 mm and 1.0 mm. The depicted footprint may measure roughly 254 mm×254 mm (10 in×10 in), yielding an areal extent of approximately 645 cm²—well in excess of the 100 cm² minimum so that an electrostatic field may be distributed uniformly across lumbar, thoracic, or extremity regions without causing localized hot spots. In other embodiments, the planform may adopt rectangular or irregular outlines tailored for cervical or extremity wraps, yet the dashed outline in FIG. 5B may indicate that any perimeter shape maintaining comparable area can furnish equivalent therapeutic performance.

A narrow, unshaded border surrounding copper plate 502B in FIG. 5B may denote an overlying dielectric layer, which is not called out by a reference numeral in this figure set but which may comprise a polymethyl methacrylate sheet. The dielectric layer may extend beyond the copper on all sides to create a non-conductive overhang that enforces creepage distance when the laminated assembly is mounted within a cushion or chair, forming a dielectrically encased copper plate electrode. Adhesive bonding between the copper and acrylic may be achieved using a UV-curable, low-shrinkage epoxy so that no air gaps exist, thereby minimizing corona onset voltage and providing a smooth patient-contact surface.

Turning to FIG. 5A, the side-A elevation 502A reveals that the copper plate is centrally embedded and that its edge terminates flush with the lateral faces of the acrylic cover. A visible longitudinal slot along the mid-height of 502A may accommodate a medical-grade, double-insulated high-potential lead (unnumbered in this view) that brings stepped-up pulses from the transformer secondary to the copper conductor. The slot may be milled to a depth that is slightly less than the acrylic thickness, permitting the cable to exit orthogonally without protruding beyond the exterior surface. After cable insertion, the slot may be potted with silicone rubber so that dielectric integrity exceeds 15 kV/mm.

FIG. 5A also emphasizes overall thickness. The acrylic may measure about 5 mm, and the combined composite—copper plus dielectric—may remain under 6 mm so that the assembly fits standard massage-table recesses. A 45-degree bevel at the top-left corner of 502A may demonstrate a laser-cut chamfer process; such bevels may minimize sharp edges, facilitate wipe-down cleaning, and reduce stress concentration that could otherwise crack the acrylic under accidental drops.

Moving to the opposite elevation, FIG. 5C depicts side-B view 502C, which may mirror 502A except for the addition of a small circular aperture near the upper edge. The aperture may accept a recessed brass binding post or spring-probe port through which maintenance technicians can verify copper continuity without removing the dielectric shell. When incorporated, the port may be sealed with an O-ring-backed plug so that normal operation remains entirely non-contact. The symmetry of 502C with respect to 502A may allow the electrode to be installed either face up or face down during field service, simplifying inventory logistics.

Surface shading in FIG. 5C may further clarify that the dielectric encapsulation completely envelopes both faces and all edges of the copper; thus, current can couple into a patient only capacitively, never conductively. This arrangement aligns with a safety intent, which calls for displacement-current return paths rather than galvanic pathways, and ensures that the practitioner may modulate field concentration merely by touching the patient while standing on grounding plate 1002 elsewhere in the system.

In some embodiments, the laminated electrode assembly may incorporate fiducial markers—such as shallow laser-etched crosses—in two diagonally opposite corners, hidden beneath the acrylic. These markers may aid automated pick-and-place robots during assembly into furniture-grade pads, but they remain electrically benign. Likewise, copper plate 502B may be subdivided into discrete zones by photo-etched isolation gaps, a feature that, although not shown in FIGS. 5A-5C, may support multiple, independently addressable treatment electrodes.

Material selection for copper plate 502B may fall to either oxygen-free high-conductivity (OFHC) copper, valued for minimal grain boundary oxide, or electroless-plated aluminum where weight reduction is paramount. The plate may be degreased in alkaline solution, micro-etched, and plasma-cleaned immediately before epoxy application so that adhesion exceeds 1,000 psi shear. The acrylic cover may be produced from optical-grade PMMA, polished on both sides to achieve a surface roughness below 0.4 μm Ra, thereby offering comfortable skin contact and visual inspection of the underlying conductor in quality-control scenarios.

Arrowheads printed at the ends of the length line in FIG. 5B may signal preferred direction for current flow during the lamination press cycle; maintaining consistent copper-grain orientation across batches can minimize warpage. Smaller arrows on FIG. 5A may identify loci where dielectric thickness is verified with ultrasonic calipers, demonstrating process-control checkpoints.

During use, each high-potential pulse introduced via the hidden cable may charge copper plate 502B relative to earth ground, establishing a displacement field that penetrates the dielectric and couples into the patient's tissue. Because dielectric thickness is fixed and acrylic permittivity is stable over temperature, field strength at the skin interface may scale linearly with plate voltage, which the practitioner controls through amplitude knob 904 and system-voltage adjuster 108. The extensive area of 502B distributes charge so that surface field density remains below 10 kV/m², thereby preventing micro-arcing even when the practitioner concentrates energy at a localized region by touch.

Cleaning and biocompatibility considerations are accommodated by the non-porous, chemically inert PMMA shell. A simple wipe with 70% isopropanol may remove oils without clouding, and the acrylic may be tinted very lightly (for example, smoke-gray at 15% opacity) so that fingerprints are less visible while still allowing visual inspection for delamination.

Collectively, FIGS. 5A-5C disclose the physical architecture, dimensional targets, and safety-oriented edge treatments of an electrode that, when coupled to the high-potential output of the step-up transformer, may capacitively deliver pulsed electrostatic fields.

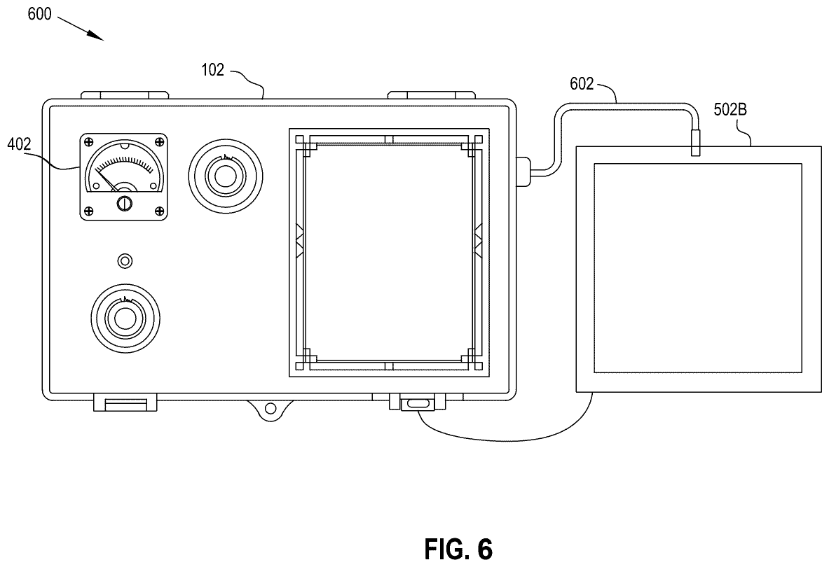

In some aspects, referring to FIG. 6, therapy-interface assembly 600 may illustrate how an electrostatic-field therapy apparatus routes high-potential output pulses from an internal pulse-gated electrostatic-field generator toward a laminated electrode that is positioned proximate a patient. A portable ARC Reactor enclosure 102 may be shown at the left of the drawing, and a laminated copper plate 502B may be located at the right. A flexible, strain-relieved high-potential lead may emerge from the enclosure and terminate at a keyed connector 602 that is potted within the dielectric layer overlying the copper plate. In many aspects, this single cable path may serve as the only conductive bridge between the step-up transformer secondary housed inside ARC Reactor enclosure 102 and the external electrode assembly, thereby helping to preserve at least 5 kV of creepage and clearance distance between user-accessible surfaces and energized conductors, for example.

In some implementations, the ARC Reactor enclosure 102 may be a clamshell-style case fabricated of powder-coated aluminum or impact-resistant polymer. Although internal or constituent components such as the power source, a pulse-rate generator, a solid-state switching stage, and a step-up transformer are not explicitly visible in FIG. 6, ARC Reactor enclosure 102 may physically protect those modules and provide mechanical anchoring for the feed-through fitting that launches the high-potential lead. A molded strain-relief grommet may be compression-fitted into a chassis aperture on the right flank of ARC Reactor enclosure 102, thereby anchoring the cable jacket to resist at least 50 N of pull force. In other aspects, an internal ferrule or cable-clamp may further immobilize conductors relative to the transformer's secondary terminal, helping to meet the wiring-schedule guidelines that require secure retention during transport.

In various aspects, the cable extending from ARC Reactor enclosure 102 toward connector 602 may be classified as a medical-grade, double-insulated, high-potential lead. The outer sheath may be formed of silicone elastomer having a dielectric withstand rating of no less than 10 kV rms. Under the sheath, a braided conductive-polymer shield may optionally be connected to chassis ground to reduce radiated electromagnetic interference, although other embodiments may omit the shield to maximize flexibility. A stranded tinned-copper core of 18 AWG or finer may carry the high-potential pulses; an integral Kevlar® ripcord may be co-extruded with the conductor to arrest elongation during repeated bending cycles. The free length of the cable may be selected between 0.3 m and 1.0 m so that the copper plate 502B can be positioned beneath a patient while the enclosure remains safely off-body on a cart or bedside shelf.

Connector 602 may be realized as a twist-lock, keyed, single-pole receptacle assembly that includes complementary plug and jack halves. The enclosure-side plug may incorporate radial bayonet lugs that mate with a quarter-turn receiver on the plate-side jack, thereby enabling tool-free engagement while resisting accidental decoupling under patient movement. In many aspects, both halves may be molded from a high-impact polyetherimide material that retains dimensional stability at sterilization temperatures up to 120° C. Internally, recessed spring contacts may maintain greater than 5 kV of creepage distance from the outer housing, and an optional silicone boot may interface to a peripheral groove on the jack to establish an IP-67 environmental seal. A color-coded collar, for instance red to denote “high voltage,” may give visual feedback to technicians during setup.

In certain aspects, the plate-side half of connector 602 may be embedded flush with the dielectric layer that overlies copper plate 502B. The dielectric may comprise a polymethyl methacrylate sheet at least 5 mm thick, as elsewhere discussed, and may be adhesively bonded to the copper using optical-grade epoxy. A countersunk cavity in the acrylic may house the rear shell of the jack so that no metallic structure protrudes above the dielectric surface that faces the patient. This geometry may help ensure that displacement current enters the patient only through capacitive coupling rather than through an exposed conductor. During manufacture, potting compound—such as a UV-curable low-shrinkage urethane—may fill any void around the jack barrel, locking the connector in place and sealing out perspiration or sanitizing fluids.

Copper plate 502B may be depicted in FIG. 6 as a planar electrode that is dimensioned to provide an areal extent of at least 100 cm². The drawing shows a rectangular outline; however, in other aspects, elliptical or contoured geometries may be substituted to match specific anatomical regions. The copper thickness may be established between 0.5 mm and 1.0 mm so that the plate retains sufficient rigidity for manual handling yet remains thin enough to conform slightly to upholstered surfaces. A through-hole or blind threaded insert on the underside of the plate may accept the threaded stud of connector 602, forming the electrical junction between the high-potential cable and the electrode. Solderless crimp lugs or silver-filled conductive epoxy may be employed to reduce contact resistance, although alternative fastening methods may be applied.

In other aspects, the dielectric covering the underside of copper plate 502B may project beyond the copper perimeter to define a protective lip that prevents inadvertent edge exposure. A beveled 45-degree chamfer around the dielectric rim may be produced by laser cutting, thereby rounding sharp corners and improving patient comfort. Epoxy bond-line thickness may be controlled to under 0.2 mm to minimize dielectric losses and field distortion. For installations where additional abrasion resistance is desired, a polyurethane over-laminate may be added to the patient-facing surface without materially altering capacitive coupling characteristics.

In several aspects, the positional relationship captured in FIG. 6 may indicate that the laminated electrode assembly can be fully detached from ARC Reactor enclosure 102, facilitating field servicing or rapid swap between multiple plate geometries. Such modularity may also permit an upgraded electrode that contains multiple discrete conductive zones. The twist-lock action of connector 602 may allow quick interchange while maintaining the stringent creepage distance required when the system is energized.

For instance, during a typical therapy session, ARC Reactor enclosure 102 may rest on a treatment-cart shelf, while copper plate 502B may lie beneath a patient's lumbar region. Once connector 602 is engaged, the practitioner may energize the system from a low-voltage supply between 3 V and 14 V. Each gating pulse generated internally may propagate through the high-potential lead into copper plate 502B, where the dielectric layer capacitively delivers the resulting electrostatic field to the patient's tissues. When the practitioner simultaneously stands on a resistive grounding interface not shown in this figure, displacement current may return safely through the practitioner to earth ground, intensifying the field at hand contact points, for example.

In many aspects, the materials and geometries depicted in FIG. 6 may be adjusted without departing from the scope of the disclosed apparatus. Connector 602 could be replaced with a threaded LEMO-style push-pull plug; ARC Reactor enclosure 102 could route the cable through a right-angle bulkhead; or copper plate 502B could employ an aluminum conductor with copper cladding to reduce cost. Similarly, the dielectric may be fabricated from polycarbonate or fluoropolymer sheets of comparable thickness to polymethyl methacrylate if greater impact resistance or chemical inertness is desired.

In some embodiments, the detachable-cable topology illustrated in FIG. 6 may be used across a variety of industry and technological domains, including sports-medicine clinics, veterinary care facilities, aerospace crew-recovery stations, and even consumer wellness furniture, thereby demonstrating the broad adaptability of the underlying pulsed electrostatic-field platform. It shall be noted that each of these disparate environments may impose unique regulatory or sterilization requirements, and the twist-lock connector 602 may be re-engineered with alternate shell materials, contact platings, or ingress-protection ratings to satisfy such sector-specific mandates without altering the fundamental capacitive-coupling mechanism already disclosed.

Firstly, unlike conventional electrotherapy devices that rely on disposable adhesive pads susceptible to drying out or causing skin irritation, the connector 602 and copper plate 502B arrangement may enable a non-contact interface that avoids consumables altogether, thus reducing recurring operating costs. Secondly, in some embodiments, the quick-exchange capability afforded by connector 602 may shorten turnaround time between therapy sessions, boosting clinical throughput while minimizing patient down-time. Thirdly, because the single-point hand-off localizes all high-potential conductors within a shielded cable run, electromagnetic emissions may be significantly lower than those of legacy spark-gap coils, thereby easing electromagnetic-compatibility testing.

In summary, as illustrated in FIG. 6, the modular electrode concept may further streamline future upgrades, such as integrating an array of discrete conductive zones selectable via a multiplexed connector shell, providing a clear pathway toward smart, software-addressable treatment maps in subsequent product generations. A multi-region electrode may be configurable to augment or replace the single-plate design.

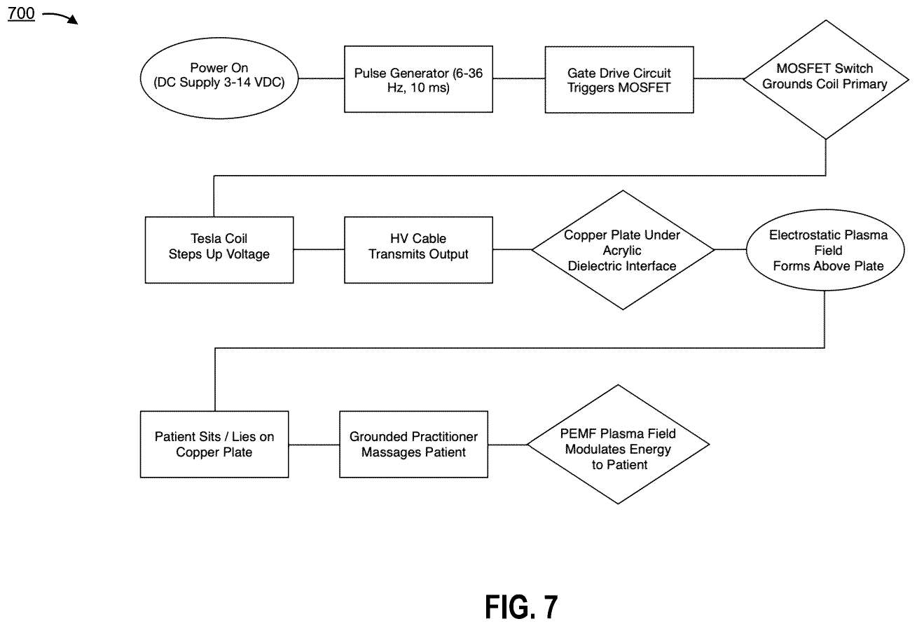

FIG. 7 depicts an operational flow diagram, according to various examples.

In some aspects, referring to FIG. 7, therapy-sequence diagram 700 may outline an illustrative operational flow that coordinates electronic subsystems and human interactions to deliver pulsed electrostatic-field energy through capacitive coupling. Dashed arrows may represent signal or energy transfer, whereas solid arrows may denote a chronological progression from a power-up event through completion of a practitioner-guided treatment cycle.

In certain aspects, the process may begin at power-on block “Power On (DC supply 3-14 VDC).” Here, a direct-current power source contained within an ARC Reactor enclosure may regulate an input voltage that may range from approximately 3 volts to 14 volts, consistent with the low-voltage envelope described elsewhere. The power source may include a buck-converter module, a rechargeable lithium-ion pack, or a mains-derived rectifier, any of which may charge an energy-storage capacitor bank that supplies peak current to a switching stage.

As shown by the next block “Pulse-rate generator (6-36 Hz, 10 ms),” energizing the low-voltage rail may activate a pulse-rate generator configured to produce gating pulses. In many implementations, the pulse-rate generator may incorporate dual 555-type timer integrated circuits that provide independent control of frequency and pulse width. A selectable repetition rate may be adjustable within approximately 6 hertz to 36 hertz, while an exemplary pulse width may be about 10 milliseconds. Other embodiments may substitute a micro-controller or FPGA-based timing core that executes an adaptive frequency sweep, for instance.

In various aspects, the gating waveform may proceed to block “Gate Drive Circuit Triggers MOSFET.” A gate-driver stage may level-shift and buffer the timing pulses so that a power transistor—such as an N-channel MOSFET rated beyond 600 volts drain-to-source—receives sufficient gate-source voltage to transition rapidly between conduction states. Optional galvanic or opto-coupler isolation may separate low-voltage timing logic from the high-current domain, helping maintain creepage distances required for medical safety.

As indicated by the decision diamond “MOSFET Switch Grounds Coil Primary,” each gate-drive edge may permit the MOSFET to connect the primary winding of a step-up transformer momentarily to circuit ground. When conduction occurs, energy previously accumulated in the capacitor bank may discharge through the primary, converting low-voltage, high-current energy into high-potential, low-current pulses across the secondary winding. Conversely, when the MOSFET is non-conductive, primary current may cease, and the transformer magnetic field may collapse.

In several aspects, the block “Tesla Coil Steps Up Voltage” may represent an ignition-style transformer whose secondary can develop on the order of several thousand volts per pulse. The transformer core may be potted in silicone elastomer to suppress corona discharge, and compliant standoffs may mechanically decouple the unit from enclosure vibration. Magnetic flux density generated during each pulse may fall within approximately 0.5 gauss to 3 gauss at a 2 centimeter distance in certain optional embodiments.

Next, block “HV Cable Transmits Output” may show that the elevated potential propagates along what is preferably a medical-grade, double-insulated high-potential cable terminated by a strain-relieved connector. The cable length may be selected so the electrode can be positioned beneath a patient while the generator remains safely off-body. In alternative configurations, detachable leads of different lengths may be supplied for limb-specific protocols.

The subsequent diamond “Copper Plate Under Acrylic Dielectric Interface” may depict arrival of the high-potential pulse at a planar copper electrode laminated beneath a dielectric layer—such as a 5 millimeter polymethyl methacrylate sheet. The dielectric barrier may be adhesively bonded with UV-curable epoxy that exhibits low shrinkage to preserve surface planarity. The plate may possess a thickness between 0.5 millimeter and 1.0 millimeter and an areal extent exceeding 100 square centimeters so that an adequate, spatially uniform electrostatic field develops.

Upon each pulse, block “Electrostatic Plasma Field Forms Above Plate” may illustrate that displacement current charges the electrode-dielectric interface, thereby producing a low-density ion envelope just above the dielectric. In many aspects, field strength may scale with both supply voltage and pulse width but may remain within a comfortable sensation range for human subjects. Supplemental ions present in ambient air may further facilitate plasma formation, yet enclosed chambers or low-humidity environments may require extended pulse durations for comparable effect.

Parallel to the electronics pathway, block “Patient Sits/Lies on Copper Plate” may characterize the subject acting as a second capacitor plate by positioning body tissue proximate the dielectric surface. Chair cushions, massage tables, or reclining beds may support the laminated electrode horizontally or vertically, and lightweight clothing may remain in place because coupling is primarily electrostatic rather than galvanic.

In other aspects, block “Grounded Practitioner Massages Patient” may introduce a resistive grounding interface—such as grounding plate 1002—that maintains at least one megohm resistance between a practitioner's body and earth ground. Standing barefoot or with conductive footwear on the interface may allow small displacement currents to flow through the practitioner during skin contact with the patient, yet the high resistance may prevent uncomfortable shock while still localizing field lines.

Finally, as suggested by outcome diamond “PEMF Plasma Field Modulates Energy to Patient,” simultaneous presence of the electrostatic field and practitioner contact may concentrate or steer the field into targeted anatomical regions. This localized modulation may stimulate circulation, reduce perceived pain, and promote neuromuscular relaxation. Field steering may remain under continuous manual control, permitting real-time adjustment responsive to patient feedback or tissue palpation cues, for instance.

Alternative embodiments may reorder or repeat certain blocks of therapy-sequence diagram 700. For example, adaptive control firmware may insert a diagnostic branch that measures electrode current before enabling practitioner contact, or a safety interlock may halt pulse generation if the dielectric plate is removed. In portable battery-operated units, power-on block parameters may further include a real-time estimate of remaining charge capacity so that a full therapy session can proceed without interruption.

By linking start-up procedures, pulse formation events, energy transmission paths, and human-centered interactions, FIG. 7 may demonstrate how electronic modules and ergonomic components cooperate to implement a non-invasive, practitioner-guided, pulsed electrostatic-field therapy technique.

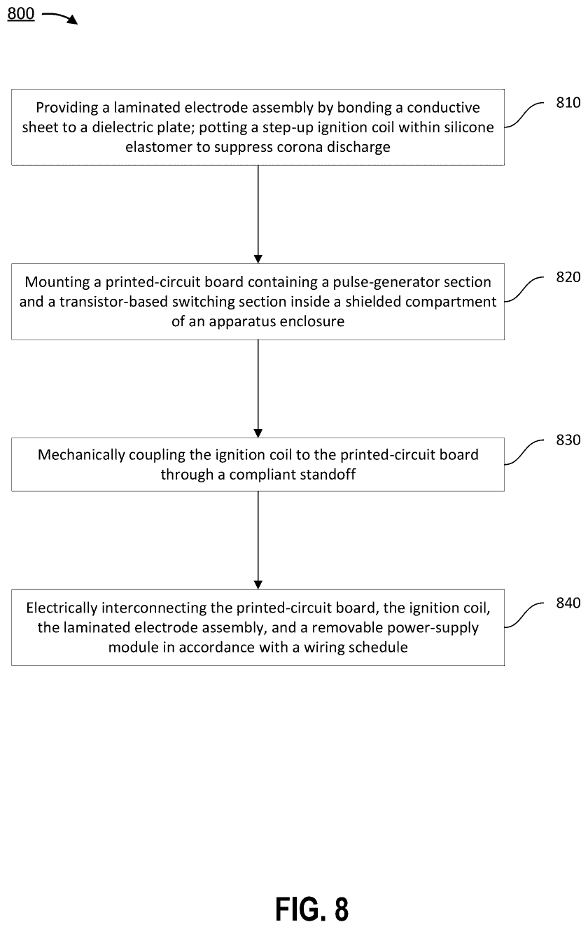

FIG. 8 illustrates a flowchart illustrating a technique for assembling an ARC reactor, according to various examples.

In some aspects, FIG. 8 illustrates method 800, which may outline an exemplary sequence of manufacturing operations that yield an electrostatic-field therapy apparatus able to generate high-potential pulses and deliver them capacitively through a laminated electrode. Dashed arrows may indicate recommended ordering, yet individual fabrication lines may rearrange or parallelize certain subprocesses without departing from the scope of the disclosure. Each block employs open-ended language so that alternative tooling choices, material substitutions, or quality-control gateways can be integrated as necessary for regulatory or scale-up considerations.

In some examples, method 800 may include providing a laminated electrode assembly by bonding a conductive sheet to a dielectric plate. In many embodiments the conductive sheet may comprise oxygen-free high-conductivity copper rolled to a thickness between about 0.5 mm and 1.0 mm, although aluminum, silver-plated brass, or copper-clad laminates may also be selected based on cost, weight, or antimicrobial properties. The dielectric plate may be a polymethyl methacrylate panel at least 5 mm thick, yet polycarbonate, polyethylene-terephthalate-glycol, or fluoropolymer alternatives may be substituted when higher impact resistance, optical clarity, or chemical inertness is desired. A UV-curable, low-shrinkage epoxy may be dispensed onto a plasma-treated plate surface, after which the copper sheet may be vacuum-laminated under 0.8 bar differential pressure to expel entrapped air. In other scenarios a roll-lamination line may hot-press the stack at 85° C. for ten minutes to accelerate polymer cross-linking. Peripheral edges of the dielectric may subsequently be laser-cut to introduce a 45-degree chamfer that may soften tactile edges while reducing corona-field concentration, for example.

In several aspects, step 820 may include mounting a printed-circuit board containing a pulse-rate generator section and a transistor-based switching section inside a shielded compartment of an apparatus enclosure. The printed-circuit board may carry dual NE555 timer integrated circuits or a micro-controller programmable to generate gating pulses within roughly 6 Hz to 36 Hz, along with a driver network that biases a power MOSFET rated no less than 600 V drain-to-source. Board material may be FR-4 or high CTI polyimide, and copper weights may be 2-oz or thicker on the power layer to accommodate pulse currents that may exceed 20 A peak. A tinned-copper shielding can may surround the timing and logic region, whereas the power stage may bolt to the enclosure floor, which may be a powder-coated aluminum chassis. Thermal interface pads of graphite or phase-change silicone may couple the MOSFET to an extruded heat sink, and a low-profile fan may direct airflow across fins to maintain junction temperatures below 80° C. EMI gaskets may line the compartment lid to meet FCC class B emissions margins. When automated optical inspection flags solder voids under the MOSFET's drain pad, rework stations may selectively reflow those joints before the board proceeds downstream.

In other aspects, step 830 may include mechanically coupling the ignition coil to the printed-circuit board through a compliant standoff. The ignition coil may be a compact, epoxy-potted transformer whose turns ratio may step 12 V primary pulses to several kilovolts on the secondary. A nylon or PEEK standoff threaded into a brass insert on the coil housing may interpose an elastomeric grommet that isolates vibration and thermal expansion mismatch between the coil mass and the more rigid PCB. Torque applied to the mounting screw may be limited—perhaps using a 0.4 N·m clutch driver—to prevent solder-pad cracking. In some implementations the standoff may include an embedded NTC thermistor so that dielectric heating within the coil can be monitored in real time; when sensed temperature exceeds an over-limit threshold, firmware may inhibit further gating pulses until the coil returns to a safe range. Optional conformal coating may be sprayed on the exposed primary pins to enhance surface tracking distance when ambient humidity is elevated, for example.

In many aspects, step 840 may include electrically interconnecting the printed-circuit board, the ignition coil, the laminated electrode assembly, and a removable power-supply module in accordance with a wiring schedule. Heavy-gauge silicone-jacket conductors—typically 16 AWG for the primary loop and 22 AWG double-insulated for the secondary—may crimp into insulated-barrel ring terminals that land on split-lock washers and PEM-staked studs to resist loosening under transport vibration. Harness drawings may specify color coding (for instance, red for V+, black for return, green/yellow for protective earth) and minimum creepage distances of at least 5 kV between adjacent high-potential pins. A keyed, twist-lock connector may route the secondary lead out to the electrode, while a polarized DC jack may accept a removable lithium-ion power pack rated 12 V nominal. For AC-mains variants, a fuse-protected IEC C14 inlet may attach via quick-disconnect spade terminals to a buck converter that provides the 3 V-14 V bus called out earlier. Serial numbers, revision codes, and test stamps may be affixed at this stage to trace each assembly through burn-in, firmware flashing, and final dielectric withstand verification, for instance.

In certain implementations, quality-control checkpoints inserted between steps 820 and 830 may verify that the pulse-rate generator section produces a gating waveform whose duty cycle does not exceed 30 percent when frequency surpasses 20 Hz. Additional verification after step 840 may confirm that displacement-current leakage through the dielectric when energized at 5 kV peak remains below 80 μA under worst-case humidity, thereby aligning with IEC-60601 patient-auxiliary current limits. Should any parameter fall outside tolerance, the wiring schedule may direct technicians to a troubleshooting flow that may swap suspect sub-assemblies without requiring total disassembly of the enclosure.

Through sequential execution of steps 810, 820, 830, and 840, method 800 may establish a reproducible, modular manufacturing path that supports both initial production and aftermarket servicing of electrostatic-therapy units. Line managers may rearrange operations—for example, integrating step 830 into a parallel cell with step 820—so long as final creepage, clearance, and functional-test outcomes conform to regulatory and performance targets.

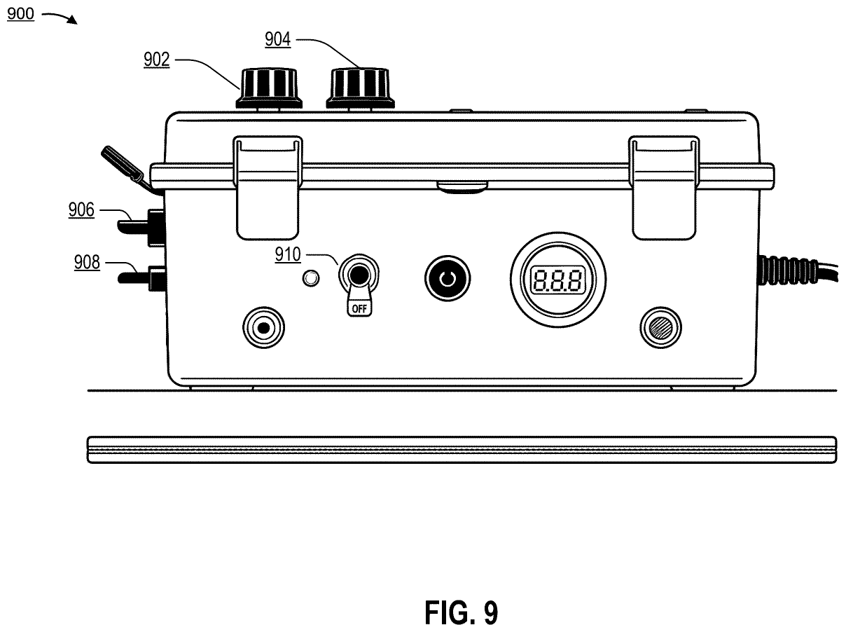

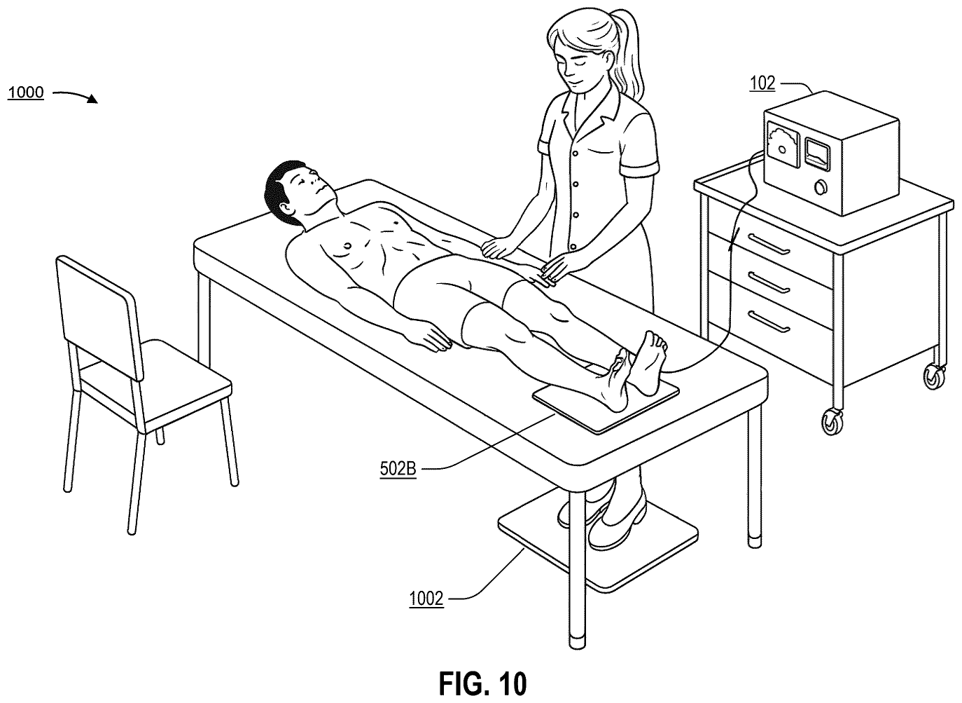

In some aspects, FIG. 9 depicts an exterior control panel that may enable an operator to energize and modulate an electrostatic-field therapy apparatus while maintaining compliance with low-voltage and mains-safety requirements. The illustrated components are positioned on a single vertical face of an ARC Reactor enclosure so that a left-to-right workflow naturally guides the user from power entry toward treatment-parameter adjustment.

In certain implementations, an AC power inlet 906 may occupy the extreme left margin of the panel. The inlet may be an IEC C14 receptacle molded from high-impact, flame-retardant polyamide rated at 250 V AC and 10 A. A built-in earth pin may bond directly to the enclosure chassis, thereby helping establish a protective-earth reference that also serves a resistive discharge path defined elsewhere in the system. A keyed orientation may prevent accidental reversal during cable insertion, and integral snap-in retention clips may facilitate tool-free replacement in the field. For regions that employ different mains connectors, AC power inlet 906 may be swapped for a country-specific CEE 7/7 or AS/NZS 3112 variant without altering downstream circuitry.

Immediately below the inlet, a fuse 908 may retain a 5×20 mm, time-delay cartridge that is sized to clear both line-to-neutral fault currents and sustained overloads arising from fan obstruction or buck-converter failure. In several aspects, the cartridge rating may be selected between 1 A and 3 A depending on market voltage so that inrush during capacitor charging does not result in nuisance trips. A spring-loaded drawer may permit rapid fuse replacement, while a transparent window may reveal filament continuity for visual inspection. By placing the fuse on the line side of the circuit, fuse 908 may satisfy medical-equipment standards that require first-line over-current protection before any patient-applied part becomes energized.

To the immediate right of the fuse holder, a power switch 910 may isolate both line and neutral conductors when toggled to the OFF position. The switch may include an internal neon pilot lamp, illuminated only when mains voltage is present downstream of fuse 908, thereby providing unambiguous feedback before additional settings are changed. A double-pole, single-throw construction may ensure full galvanic disconnection during storage or maintenance, and an IP-65 silicone gasket may seal the actuator against fluid ingress. In certain embodiments, power switch 910 may route through an enclosure-lid interlock so that the panel cannot energize the internal circuitry unless service covers are fully seated.

In various aspects, a frequency adjustment knob 902 may be positioned near the top center of the panel. Frequency adjustment knob 902 may couple through a 6 mm-diameter shaft to a 20 kΩ potentiometer housed on the reverse side of the panel, and rotation may vary the RC time constant of a first NE555 timer within the pulse-rate generator. Graduations silk-screened around the knob perimeter may correspond to a selectable repetition-rate band of approximately 6 Hz to 36 Hz, allowing practitioners to match pulse frequency to a desired neuromuscular-stimulation protocol. A detented spring washer may produce tactile clicks at commonly used setpoints—such as 7.8 Hz, 10 Hz, and 30 Hz—so that an operator may adjust frequency without diverting visual focus from the patient. Alternative embodiments may replace potentiometric control with an optical encoder interfaced to a capacitive-sense touchscreen for finer digital resolution.

Adjacent to the frequency control, an amplitude knob 904 may regulate the direct-current level delivered by a buck-converter that forms the low-voltage power source. The knob may actuate a 5 kΩ multi-turn potentiometer that feeds the converter's feedback network, providing a continuously variable output between roughly 3 V and 14 V. This range may permit field intensity to scale from gentle sensory feedback up to deeper tissue coupling. A knurled aluminum skirt with laser-engraved numeric indices may facilitate precise selection; however, in pediatric or geriatric settings, knob 904 may be substituted with a keyed switch that accesses only preset voltage levels for heightened safety.

Between knobs 902 and 904, a recessed arrow may indicate clockwise rotation as the direction that increases the respective parameter, while a thin hash-mark on each knob hub may align with the printed scale. The arrow graphics may be applied via UV-cured ink that resists abrasion from repeated sterilization wipes. In alternative designs, LED back-lighting beneath each knob skirt may pulse in synchrony with gating-pulses to provide real-time visual affirmation that adjustments are taking effect.

Although not explicitly labeled in FIG. 9, an enclosure-mounted digital meter could be included to display instantaneous bus voltage or pulse rate; however, the illustrated embodiment relies solely on tactile and incremental feedback from knobs 902 and 904. Optional retrofits may integrate a 1.3-inch OLED readout without altering the position of existing controls, preserving panel ergonomics.

The spatial grouping whereby AC power inlet 906 and fuse 908 reside at the extreme left, power switch 910 at center-left, and tuning knobs 902 and 904 on the right may intentionally create a progressive activation sequence: connect mains, verify fuse, engage power, then fine-tune therapy settings. Such arrangement may minimize accidental high-potential generation before an operator has confirmed adequate supply integrity and grounding.

From a manufacturing standpoint, the panel cut-outs for frequency adjustment knob 902, amplitude knob 904, AC power inlet 906, fuse 908, and power switch 910 may be laser-machined in a single fixture pass to ±0.05 mm tolerance. Each component may secure via threaded ring nuts with integrated locking serrations so that torque values remain stable despite vibration encountered during mobile-clinic transport. To ease servicing, quick-disconnect tab terminals may attach to power switch 910, fuse 908, and AC power inlet 906, while JST-style headers may mate the potentiometer tails for knobs 902 and 904 to the underlying control PCB.

In several implementations, the enclosure material immediately surrounding the panel may be a 2.0 mm-thick 5052-H32 aluminum sheet, powder-coated with a textured polyester resin to enhance grip and resist fingerprinting. EMI shield fingers may bridge the panel edge to a chassis frame so that rotational shafts of knobs 902 and 904 do not introduce RF leakage paths, an important consideration when the apparatus operates near diagnostic imaging equipment.

Alternative embodiments may transpose knobs 902 and 904 to a horizontal orientation for table-top units, or relocate AC power inlet 906 to a rear bulkhead when wall-mounting is preferred. Similarly, power switch 910 may be replaced by a keyed ignition-style switch to prevent unauthorized operation in rehabilitation facilities. Despite such variations, each panel arrangement may preserve the functional relationship whereby power-entry hardware precedes user-accessible treatment controls, thereby maintaining a consistent human-factors footprint across product tiers.

By presenting frequency adjustment knob 902, amplitude knob 904, AC power inlet 906, fuse 908, and power switch 910 in a concise, intuitive layout, FIG. 9 may demonstrate how an electrostatic-field therapy apparatus offers safe, user-friendly access to critical operating parameters while upholding international electrical-safety and electromagnetic-compatibility standards.

In some aspects, FIG. 10 illustrates an example clinical deployment in which a portable electrostatic-field therapy apparatus may deliver pulsed energy to a supine subject while a practitioner actively guides field concentration by touch. An ARC Reactor enclosure 102 may rest on a wheeled treatment-cart, the cart positioning the generator at approximately waist height so that front-panel controls can be accessed without interrupting hand contact with the subject. Although only the exterior of ARC Reactor enclosure 102 is visible, the housing may contain a power source, a pulse-rate generator, a solid-state switching stage, and a step-up transformer, each of which may cooperate to produce high-potential output pulses as previously described. A shielded high-potential cable (not labeled here) may exit the rear wall of ARC Reactor enclosure 102 and route toward an electrode assembly positioned beneath the subject.

In certain implementations, the electrode assembly may include a laminated copper plate 502B encapsulated beneath a dielectric sheet that is at least 5 mm thick. Copper plate 502B may lie flat upon a padded treatment table so that a patient can recline with lumbar, sacral, or lower-leg areas proximate the dielectric surface. Because copper plate 502B may present an areal extent exceeding 100 cm², field lines may emanate uniformly across a broad tissue volume, yet the dielectric barrier may ensure that no direct conduction path reaches the patient. A twist-lock connector (omitted from this drawing but shown in earlier figures) may couple the high-potential lead to an embedded jack on the underside of copper plate 502B, thereby concealing conductive hardware from the patient contact plane.

In various aspects, a resistive grounding interface may be realized as grounding plate 1002 positioned on the floor adjacent the treatment table. Grounding plate 1002 may be fabricated from a conductive-polymer matrix loaded with carbon fibers and may incorporate an internal resistor that yields a total resistance of at least one megohm between the plate surface and an earth-ground conductor. A practitioner may stand barefoot, or in conductive socks, upon grounding plate 1002 so that a high-impedance return path is established for displacement current generated by the pulsed electrostatic field. The megohm-class resistance may limit touch currents to micro-ampere levels that remain below IEC-60601 patient-auxiliary thresholds while still permitting sufficient field steering.

During a typical session, the practitioner may first activate an AC power switch on ARC Reactor enclosure 102 and adjust frequency and amplitude controls so that a repetition rate within approximately 6 Hz to 36 Hz and a bus voltage between roughly 3 V and 14 V are selected. Once settings are confirmed, the practitioner may instruct the patient to lie on the table with the target body region centered over plate 502B. A lightweight cotton drape may remain between skin and dielectric because capacitive coupling is only weakly affected by non-metallic fabrics of limited thickness.

To establish a controlled return path for displacement current, a resistive grounding interface may be shown at floor level as grounding plate 1002. Plate 1002 may comprise a carbon-impregnated elastomer layer laminated to a copper mesh bus, the composite presenting at least 1 MΩ resistance between its upper surface and an earth-ground terminal. The practitioner's shoes may incorporate conductive forefoot inserts or may be removed entirely so that socks or bare skin contact the plate directly. A flexible grounding cable 1004 may link the bus embedded in plate 1002 to a chassis stud on ARC reactor enclosure 102 or to a dedicated earth pin in a mains outlet. Cable 1004 may employ 18 AWG stranded copper conductors sheathed in silicone for medical-grade wipe-down compatibility and may terminate in crimped ring lugs captured by nylon lock-nuts to resist loosening during cart repositioning.

With the patient settled, the practitioner may stand upon grounding plate 1002, place both hands on the patient's skin, and then press a system-start button on ARC Reactor enclosure 102. Internally, the pulse-rate generator may begin issuing gating pulses that switch a MOSFET on and off; these actions may cause primary-side current pulses to flow through an ignition-style transformer, thereby producing secondary-side voltages on the order of several kilovolts. Each voltage spike may charge copper plate 502B for approximately 10 ms, after which the plate potential may collapse toward ground before the next cycle commences. The dielectric layer may permit displacement current to penetrate toward the patient's tissue, forming a diffuse electrostatic plasma envelope that may be sensed as warmth or gentle tingling.

When the practitioner's hands contact the patient, the human body of the practitioner may provide a preferential return route for displacement current because the practitioner's feet are electrically referenced to earth via grounding plate 1002. Consequently, field lines may converge toward the hand-skin interface, locally amplifying field strength in the tissue region immediately beneath the practitioner's fingertips. By sliding a hand along muscle fibers or applying light circular pressure over a joint capsule, the practitioner may dynamically steer energy deposition to regions of heightened stiffness or inflammation. Because coupling is purely capacitive, the practitioner may perceive only a mild vibratory sensation, and no galvanic conduction path may develop that could provoke involuntary muscle contractions associated with traditional TENS electrodes.

In other aspects, ARC Reactor enclosure 102 may log treatment parameters—such as start time, stop time, selected repetition rate, and pulse amplitude—into non-volatile memory so that cumulative exposure metrics can be reviewed at follow-up appointments. A thermal sensor mounted near the internal MOSFET may suspend pulse generation if component temperature exceeds a safe threshold, and an audible chime may alert the practitioner to pause hands-on activity while the system cools.

Alternative embodiments may employ grounding plate-equivalent interfaces in footwear form; for example, conductive insoles bonded to a one-megohm resistor could permit the practitioner to move freely around the treatment space without repositioning grounding plate 1002. Likewise, copper plate 502B could be replaced by multiple smaller copper pads—each selectively energized—so that bilateral limb therapies can proceed without shifting the patient.

From a workflow standpoint, FIG. 10 may demonstrate that only three physical components—ARC Reactor enclosure 102, copper plate 502B, and grounding plate 1002—need be positioned in the clinical environment. All high-potential conductors may remain shielded and strain-relieved, and the enclosure may reside safely beyond the patient reach zone. Setup time may therefore be limited to under two minutes, and tear-down may merely involve detaching the high-potential connector and wiping dielectric surfaces with isopropyl alcohol.

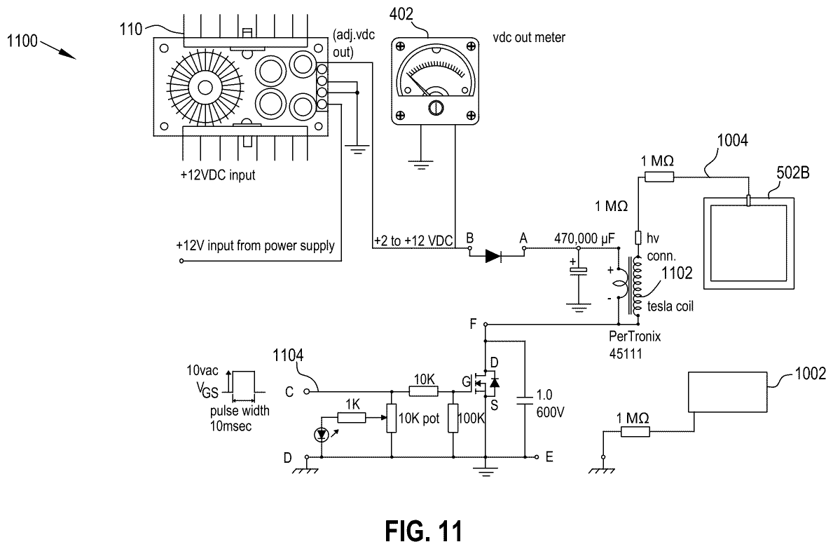

Referring to FIG. 11, in some aspects, an electrostatic-field therapy circuit. such as that shown in example circuitry 1100. may integrate multiple subsystems that cooperate to generate, condition, and route high-potential pulses toward a laminated copper electrode positioned near a subject. A low-voltage supply line, preferably marked “±12 V input from power supply”, may enter a compact switch-mode converter module, which may include integrated heat-spreading fins and an internal buck topology that, in certain implementations, may step a nominal 15-24 V wall-adapter feed down to a selectable 3 V-14 V direct-current rail. This module may further house reverse-polarity protection and soft-start circuitry so that inrush currents into downstream storage capacitance remain below one ampere, for instance.

In various aspects, the DC output of the aforementioned module may be monitored by a VC input readout 402 that is referenced to chassis ground. VC input readout 402 may include a 0-15 V scale sized for direct visual confirmation that the “power source” operates within the safe 3 V-14 V envelope discussed with respect to claim 2. A single pointer needle may deflect in real time, allowing a practitioner to correlate any amplitude adjustments to actual rail voltage without secondary instrumentation.

Down-line from the converter, node A may feed a high-capacity energy-storage capacitor 470,000 μF that, in some embodiments, may be realized by paralleled electrolytic cans configured for less than 20 mΩ equivalent series resistance. The capacitor bank may act as a buffer, sourcing peak currents on the order of tens of amperes during each pulse while limiting voltage sag at node A below ten percent of nominal. A series diode positioned between node A and the capacitor may provide polarity isolation so that stored charge cannot back-feed into secondary Tesla drive-voltage 110 during shutdown scenarios.

Node A also connects to the primary winding of coil 1102, identified in the figure as “PerTronix 45111.” In certain aspects, coil 1102 may embody a “step-up transformer,” presenting a turns ratio in the range of 1:100 and generating secondary potentials that may exceed 30 kV on open circuit. The primary may be wound with heavy-gauge enamel copper to keep inductance low, thereby permitting rapid current rise when the coil is periodically grounded by the switching stage.

In many aspects, the switching stage may be mounted on driver board 1104. A square-wave gate drive signal VGS, shown as “10 VAC pulse width 10 msec,” may arrive at board 1104 on control node C. This waveform may originate from a dual-timer pulse-rate generator similar to that depicted in earlier figures, thereby satisfying the “pulse-rate generator configured to produce gating pulses” element. A series resistor of approximately 1 kΩ, followed by an adjustable potentiometer of 10 kΩ, may form a voltage-divider network allowing practitioners to trim gate amplitude so that the MOSFET switches fully while avoiding excessive dissipation.

The central solid-state device is an N-channel MOSFET whose drain D is shown tied through node E to chassis ground. In operation, each positive VGS excursion may enhance the MOSFET channel, causing the drain-source path to conduct and thereby pulling the ignition-coil primary to ground for roughly 10 milliseconds. As the current collapses at turn-off, the coil secondary may produce a high-potential pulse that exits via a shielded high-tension cable 1004, which is a high-tension insulated cable preferably, labelled “hv cabel” in the drawing. The MOSFET package may be bonded to a heat sink (not specifically shown in this figure) so that junction temperatures remain within the thermal-management boundary conditions contemplated by claim 6; optionally, a thermistor sensor may disable the gate-drive signal if case temperature exceeds 90° C.

In certain aspects, the secondary lead of coil 1102 may terminate at a panel-mount high-potential connector that mates to grounding cable 1004. Grounding cable 1004 may route the pulses toward copper plate 502B, illustrated at the right of FIG. 11 beneath a dielectric encapsulant. Copper plate 502B may have an approximate thickness between 0.5 mm and 1.0 mm and laminated under at least 5 mm of acrylic so that displacement current, rather than conduction current, reaches the subject.

A practitioner-grounding pathway is depicted by grounding plate 1002. Grounding plate 1002 may include an internal resistance of not less than one megohm, thereby providing a controlled discharge path compliant with claim 5. When a practitioner stands upon grounding plate 1002 and simultaneously touches a patient positioned proximate copper plate 502B, displacement current may close through the practitioner's body, concentrating electric flux at the touch points. In this way, field intensity may be directed without repositioning hardware—a therapist-guided modality that aligns with system claim 14.

Intermediate node F on driver board 1104 is shown returning to ground, ensuring that high-current switching loops remain short and coaxial, which may mitigate electromagnetic interference. Additionally, a snubber capacitor of approximately 1 μF rated 600 V is wired across the drain and source pins, limiting flyback overshoot and prolonging MOSFET life. An adjustable 100 kΩ resistor connected gate-to-source may provide a default bias that turns the device off when control signals are absent.

In several aspects, the figure also notes a 470,000 μF electrolytic capacitor along the energy path; however, future figures may dissect this storage bank's mechanical mounting and bleed-down provisions in greater detail.

A dashed arrow from secondary Tesla drive-voltage 110 toward VC input readout 402 suggests that the same low-voltage rail energizes both the pulse-rate generator and the driver board's logic supply, maintaining consistent timing even as primary-side loading varies. A ground symbol VC input readout 402 indicates chassis reference, helping ensure that operator-touchable metal remains at earth potential.

Collectively, FIG. 11 may teach a complete pulsed electrostatic-field generator chain: regulated low-voltage source, adjustable dual-timer pulse-rate generator, MOSFET switching stage, ignition-style step-up transformer, high-tension delivery cable, dielectric-covered copper electrode, and resistive grounding interface. Each labelled element may be fabricated using commercially available parts, yet their cooperative arrangement, as illustrated, may achieve rapid, user-tunable pulse delivery suitable for non-invasive neuromuscular stimulation while maintaining electrical isolation consistent with medical safety guidelines.

In many aspects, enclosure 102 may be machined from 2.5 mm-thick 5052-H32 aluminum plate held to ±0.05 mm dimensional tolerance on all orthogonal edges, and then formed on a CNC press brake so that each corner bend retains a 4 mm inside radius, thereby reducing localized stress risers that could propagate cracks under repeated transport vibration. Inside surfaces may receive a chromate conversion coating prior to powder-coat application, elevating salt-spray resistance beyond 1000 h, whereas exterior faces may be textured with a 70 μm-thick polyester resin providing ≥80 Shore D hardness so that minor abrasions remain visually inconspicuous in mobile-clinic environments. A pair of M4 threaded PEM studs may be spot-welded to the inner right wall to anchor a heat sink that spans the full height of a MOSFET mounting flange, and four silicone-in-steel vibration grommets may suspend the entire electronics tray from the enclosure shell to isolate 25-200 Hz road-induced vibrations when the system is transported by vehicle.

Sub-floor rails molded of glass-filled polyamide may retain sliding battery pack cartridges so that an operator can load packs axially from the rear with a single-hand push until spring-biased detents click into a machined recess. A keyed microswitch may verify full pack insertion before a solid-state relay bridges the pack to the DC bus, introducing electronic interlocking redundancy should a user attempt to energize the system with an incompletely latched pack. In certain embodiments, a Hall-effect latch may replace the microswitch, thereby eliminating mechanical wear points and supporting an IP-67 seal when the unit is configured for military-field deployments.

Ignition coil 112 may feature a double-stacked bobbin wound with 0.4 mm square copper magnet wire insulated to class F (155° C.) and then vacuum-potted in a two-part silicone elastomer exhibiting 25 kV/mm dielectric strength. A ferritic steel core may undergo manganese-zinc doping to maximize permeability at the 5-40 kHz magnetizing frequency that results from a 10 ms pulse width; core loss measured per IEC 62024 may remain below 40 mW/cm³ when driven at 14 V on the primary. The secondary lead exit may incorporate a PTFE sleeve heat-shrunk to the silicone encapsulant so that no sharp polymer meniscus intrudes into the creepage path, reducing partial discharge risk at altitudes up to 3000 m.

Copper plate 502B may be laser-cut from C11000 alloy and then planished to maintain out-of-flatness within 0.07 mm across its 254 mm diagonal. After planarization, a 2 μm electroless-nickel barrier may be over-plated with 0.4 μm immersion gold to inhibit copper ion migration into the adhesive interface over time. The dielectric sheet bonded to copper plate 502B may be a cast polymethyl-methacrylate meeting ISO 7823-1, having a tensile strength of 75 MPa and an Izod-notched impact resistance of 15 kJ/m². A laser-cut periphery may carry a 45-degree chamfer 1 mm deep; the root of that chamfer may be polished with a 3 μm diamond compound to Ra≤0.3 μm so that the plate can be wiped with lint-free cloths without snagging fibers. In alternate configurations, the dielectric topcoat may be a co-molded thermoplastic polyurethane layer 0.25 mm thick that elevates scratch resistance while preserving field transparency, enabling use in chiropractor offices where repeated instrument contact could abrade untreated acrylic.

Grounding plate 1002 may comprise a 3 mm stainless-steel substrate (such as AISI 304) coated on the upper face with a 2 mm layer of carbon-impregnated thermoplastic elastomer injection-molded directly onto the metal. An embedded spiral trace of resistive ink printed on a polyimide film may supply a calibrated 1 MΩ pathway between plate and an IEC 60320-compliant ground conductor. Edge-welded studs may allow the ground strap to be removed without delaminating the elastomer, facilitating periodic high-pot isolation testing. In other embodiments, grounding functionality may be incorporated in footwear, such as conductive-polymer soles offering 1-5 MΩ resistance may interface with an ESD floor grid, eliminating trip hazards posed by standalone plates.

Cable terminations at connector 602 may utilize crimp-and-solder “belt-and-suspenders” joints: first a Mil-Spec crimp forms a cold-weld gas-tight connection, after which low-temperature tin-lead solder back-fills interstitial voids, ensuring vibration resilience exceeding 10 g RMS when tested to MIL-STD-810H, Method 514.8. A silicone potting gland molded around the rear shell of connector 602 may anchor aramid tensile members within the cable so that strain never transfers to crimp barrels, and the gland may be over-filled by 1 mm relative to the acrylic recess such that potting compound forms a positive meniscus, providing wipe-clean hygiene.

Control firmware may implement a PID loop that samples plate voltage via a capacitive divider coupled to a 24-bit sigma-delta ADC; if sensed amplitude deviates more than ±2% from the operator's setpoint—owing perhaps to temperature-driven coil impedance drift—the MCU may modulate duty cycle in 0.5% increments until the error band is recentered. Temperature sensor data sampled adjacent to MOSFET drain pad may feed a second loop that linearly derates duty cycle beginning at 70° C. and fully inhibits pulses at 85° C., thereby creating a self-healing thermal fail-safe independent of practitioner attention.

For rugged environments, an alternate modular configuration may enclose copper plate 502B in a hinged ABS frame carrying four rare-earth magnets that mate to steel inserts on the treatment table. Such coupling may permit rapid plate removal for disinfection yet still ensure repeatable placement accuracy within ±5 mm of anatomical landmarks previously mapped in electronic patient records. A spring-loaded latch may prevent vertical lift unless a button detent is pressed, providing a mechanical child-safety lock.

When transporting the system by air, ARC reactor enclosure 102 and both plates may stow in a roller case whose foam cavities compress around each component; viscoelastic urethane foam having Shore 00 hardness of 20 may damp impact energies up to 150 J. A pressure-equalization valve rated IP-68 may prevent altitude-induced vacuum that could damage the acrylic-to-copper bondline. For remote humanitarian deployments where mains power is intermittent, a fold-out 60 W solar panel may plug into a charge controller built into the battery pack, allowing eight full 20-minute therapy sessions after six hours of peak sun—an energy-autonomy profile unattainable with conventional magnetic-coil PEMF benches exceeding 200 W standby draw.

Calibration may involve placing a calibrated B-field probe 15 mm above the dielectric while the firmware sweeps frequency; an on-screen wizard may adjust gate-drive amplitude so that peak flux at 10 Hz equals 2.0 gauss±0.3 gauss. Field uniformity may then be verified by translating the probe laterally; any zone deviating more than 10% may trigger a soft-limit in firmware that confines duty cycle within a reduced operational window until service replacement of the coil is scheduled.

Multiple fail-safe mechanisms may coexist. A latching e-stop on the cart handle may shunt the MOSFET gate to ground through a 100Ω resistor, dumping stored energy in <5 ms. A redundant mechanical interlock under the cart shelf may sever AC mains to the buck converter if the cart topples more than 45°, a feature especially useful aboard mobile ambulances negotiating uneven terrain. Should both e-stop and mains cut-off fail, an inline thermal fuse rated 100° C. embedded within coil 112 may permanently open, preventing continuous overheating.

From an industrial-applicability standpoint, the same capacitive-coupling principle may treat equine tendon injuries by swapping copper plate 502B for a flexible copper-mesh sleeve that wraps around the fetlock, while grounding plate 1002 may be dimensioned as a hoof mat. In factory robotics, anti-static conditioning of sensitive electronic assemblies may leverage plate arrays energized at sub-sensory voltage levels to reduce triboelectric buildup on operator garments. These divergent use cases illustrate how detailed mechanical descriptions—such as detachable connector 602, modular electrode geometries, and controlled displacement-current paths—support a broad legal scope.

Technicians may tune system behavior post-installation by inserting 0.1 mm aluminum shims between heat sink and enclosure wall, modifying thermal conductivity such that fan duty cycle remains below 40% in 40° C. ambient. In research laboratories, an auxiliary BNC port may export coil-primary current waveforms to an oscilloscope, letting investigators correlate cell-culture responses to precise field signatures.

Collectively, the enhanced mechanical robustness, modularity, redundant safety layers, and calibration pathways described above provide the skilled artisan with clear guidance for fabricating, integrating, and adapting the disclosed electrostatic-field therapy apparatus across diverse clinical and industrial contexts while preserving the fundamental capacitive-coupling mechanism.

By depicting ARC Reactor enclosure 102 as the field generator, copper plate 502B as the capacitively coupled electrode, and grounding plate 1002 as the resistive discharge path, FIG. 10 may convey how pulsed electrostatic therapy can be delivered non-invasively, with practitioner-guided localization, and with minimal consumable materials. This arrangement may provide a power source, a pulse-rate generator, a transistor-based switching stage, a step-up transformer, an electrode-dielectric assembly, and a resistive grounding interface that collectively concentrate an electrostatic field at practitioner-selected regions of a subject's body.

In some aspects, an electrostatic-field therapy apparatus may integrate a multi-stage hardware architecture that begins with a low-voltage power source and culminates in a dielectric-covered electrode that capacitively delivers pulsed fields to living tissue. A regulated buck-converter module may accept 90-264 V AC at an AC input receptacle 306 and may generate a bus that is operator-selectable between about 3 V and 14 V DC. For mobile deployments, a removable 4-cell lithium-ion pack rated 12 V nominal may dock through a keyed DC barrel so that the same downstream electronics continue to operate without firmware reboot. Parallel Schottky-or-diode ORing may permit seamless switchover between pack and mains while a Coulomb-counter ASIC logs net charge transfer for battery-health analytics.

A pulse-rate generator may reside on a four-layer printed-circuit board whose logic section is isolated from the power section by an air gap greater than 8 mm. In a baseline embodiment, two NE555 timers may be wired in cascaded astable-monostable topology; turning frequency adjustment knob 902 may vary a first RC network so that repetition rate covers roughly 6 Hz-36 Hz, while rotating amplitude knob 904 may trim the bus voltage, indirectly modulating electrostatic-field strength. Because each timer has its own timing capacitor and potentiometer, a practitioner may fine-tune duty cycle independently of frequency, which may be difficult to replicate mentally and thus underscores that the process cannot be performed in the human mind. A micro-controller alternative may sample the analog pots through 12-bit ADC channels and render numeric feedback on a capacitive-sense touchscreen; an RTOS task may then update a PWM waveform that drives the same gate-driver transistor.

Downstream of the timing core, a gate-driver network may incorporate a totem-pole pair of MOSFETs that translate 0-5 V logic swings into 0-12 V gate excursions with sub-100 ns rise/fall edges. A primary power MOSFET—such as an STP50N60DM6 rated 600 V, 47 A—may mount on a graphite-filled or graphite-based thermal pad that in turn mates to an extruded aluminum heatsink bonded to the enclosure wall; a 30 CFM axial fan under PWM control may engage when board thermistor readings exceed 55° C. The MOSFET's drain may connect to one terminal of a step-up ignition coil potted in silicone to suppress corona per method step “potting,” while the other terminal of the coil primary may return to the main VDC rail through pulse-storage capacitor bank 114A/114B (˜470000 μF). Each capacitor may be bolted to copper bus bars with spring washers so that ripple currents up to 40 A rms can circulate without overheating, thereby enabling the conduction path that periodically switches current from the power source.

The ignition-style transformer's secondary may deliver 3-7 kV peak pulses into a double-insulated silicone cable routed through a strain-relieved bulkhead fitting. A keyed twist-lock connector 602 may couple the cable to laminated copper plate 502B, which may comprise a 0.7 mm-thick copper sheet measuring 254 mm×254 mm (>100 cm²) adhesively bonded to a 5 mm polymethyl-methacrylate plate with UV-curable epoxy under vacuum lamination. Laser cutting may introduce a 1.5 mm-deep, 45-degree chamfer around the acrylic perimeter so that edge electric-field intensification is mitigated and patient comfort is enhanced. A recessed brass stud hidden beneath the dielectric may accept the connector pin; thus, no conductive surface is exposed to the patient, and the dielectric layer capacitively conveys displacement current into the body region atop the plate.

To furnish a defined return path, a grounding plate 1002 may embed a carbon-filled elastomer that presents approximately 1 MΩ to the green-yellow protective-earth conductor. When a practitioner stands on plate 1002 while simultaneously touching the patient, field lines may preferentially collapse through the practitioner's hands. Because the return impedance is several orders of magnitude higher than a direct wire, patient-auxiliary current is limited below 80 μA, far under IEC-60601 maximums, and the safety feature is inherently electronic rather than reliant on subjective human control, showing a technical improvement not performable mentally.

An aluminum suitcase enclosure may be powder-coated inside and out and partitioned by a 1.5 mm thick, RF-gasketed shield wall: logic on one side, high-potential on the other. Cable trees may follow a wiring schedule that maintains ≥8 mm creepage between any conductor carrying >1 kV and a chassis seam. A micro-controller may log session metadata—start/stop time, repetition rate, pulse amplitude—to a NOR Flash device, while a BLE module may periodically upload these packets to a companion mobile application. Over-the-air firmware updates may stream AES-encrypted binaries that adjust PWM limits or the adaptive frequency-sweep LUT without removing enclosure screws.

During manufacture, an operator may first bond copper to acrylic, then pot the coil, then mount and standoff-isolate the coil to the PCB, and finally interconnect sub-assemblies per color-coded harness drawings. Automated functional test may trigger a firmware routine that enforces <30% duty cycle above 20 Hz; if limits are exceeded, the MCU may blank the gate-driver PWM and illuminate a red fault LED, a control action that cannot be reliably executed by a human diagnostician without electronic sensing.

In certain illustrative, non-limiting embodiments, a step-up transformer may exhibit a primary inductance L of approximately 3 millihenries (3 mH). When the primary is driven at a rail voltage V of 12 volts (12 V) for a conduction interval t of 10 milliseconds (10 ms), the instantaneous primary current I can reach about 40 amperes (40 A) according to the relation I=(V×t)/L. The magnetic field thereby stores energy E, given by E=0.5×L×I², amounting to roughly 2.4 joules (2.4 J). Via a representative 1:100 turns ratio, this energy is reflected to the secondary and may produce an open-circuit crest voltage approaching 30 kilovolts (30 kV). Where the secondary is terminated by a predominantly capacitive load C_load of about 100 picofarads (100 pF)—typical of the patient-electrode interface—the high-potential pulse collapses in approximately 8 microseconds (8 μs) and forces a displacement-current transient of roughly 37 milliamperes (37 mA) through the treatment region. All numerical values are exemplary and do not limit the claimed scope.

In an exemplary, non-limiting construction, a light-emitting diode (LED) fabricated from aluminium-indium-gallium-phosphide (AlInGaP) and emitting at approximately 635 nanometres (635 nm) may be connected between control nodes C and D and biased at about 7 milliamperes (7 mA) by the same 1-kilohm (1 kΩ) series resistor that conditions the gate-drive waveform. Because the LED load is purely resistive, it introduces less than 0.1 percent duty-cycle error even at the minimum contemplated repetition rate of 6 hertz (6 Hz), thereby furnishing immediate optical confirmation of gating activity whenever the high-tension output cable is disconnected. The indicator channel is optional and should not be construed as limiting.

To moderate drain-to-source overshoot, a polypropylene-film capacitor having a capacitance C_snub of approximately 1 microfarad (1 μF) and a working voltage of 600 volts DC (600 V DC) may be mounted within roughly 15 millimetres (15 mm) of the MOSFET package so as to shunt its drain to its source. Together with an estimated transformer leakage inductance L_leak of about 25 microhenries (25 pH), this placement forms a critically damped RLC network that limits the first overshoot peak to less than 1.2×V (where V is the rail voltage). The selected dielectric exhibits a loss factor tan δ≈0.006, keeping self-heating below 5 degrees Celsius (5° C.) during continuous operation at the maximum 36 Hz pulse rate. Component values and spacings may be scaled as design requirements dictate.

In some exemplary embodiments, the coil-primary return, the MOSFET drain, and the snubber-capacitor upper terminal may converge at a single stitched copper pour (node F) executed in 70 micrometres (70 μm) of copper on the printed-circuit board. Orthogonal via fences placed on either side of the pour can confine the high-di/dt loop area to approximately 60 square millimetres (60 mm²), thereby reducing common-mode radiated emissions by about 14 decibels (14 dB) relative to an unsegmented ground plane of equal copper weight. Alternative grounding geometries may be employed without departing from the inventive concepts.

To soften the practitioner's tactile perception of each pulse, the series resistor R_gnd terminating grounding plate 1002 may be selected to be approximately 1 megohm (1 MΩ). When combined with a representative patient-to-electrode capacitance C_patient in the range 70-150 picofarads (70 pF≤C_patient≤150 pF), the resulting first-order discharge time constant τ (tau), given by τ=R_gnd×C_patient, falls between about 70 microseconds and 150 microseconds (70 μs≤τ≤150 μs). This intentional delay blunts the ionic-current component of the return path and mitigates the transient “sting” sometimes reported with faster collapse rates. Other impedance values or network topologies may be substituted to obtain comparable therapeutic or ergonomic results.

Because electrostatic-field magnitudes on the order of kilovolts cannot be intuited or safely timed by human cognition, the disclosed apparatus provides concrete circuitry—timers, drivers, isolation gaps, and dielectric interfaces—that physically embody each claimed functional block. The adaptive sweep algorithm, the thermal interlock, and the displacement-current steering all rely on real-time sensor sampling and closed-loop electronic control, operations that cannot be accomplished in the human mind with equal speed, repeatability, or safety, thereby underscoring the technical character and patent-eligibility of the system.

The PEMF therapy apparatus described herein may be powered by a low-voltage direct-current supply—preferably a removable, rechargeable lithium-ion pack rated at a nominal 12 V, but alternatively an external supply delivering between about 3 V and 14 V—and that supply may feed an energy-storage capacitor bank so that brief, high-current pulses can be produced without sag. A pulse-rate generator implemented either with dual NE-555 timer integrated circuits or with a micro-controller is separately adjustable for frequency and pulse width, the selectable repetition rate lying within roughly 6 Hz to 36 Hz. Gating pulses from the generator drive the control terminal of at least one MOSFET in a solid-state switching stage; the MOSFET is rated for at least 600 V drain-to-source and is bonded to a graphite thermal pad attached to a heat sink that may be augmented by a fan and protected by a temperature sensor configured to disable the generator if an over-temperature condition is detected. The MOSFET conduction path periodically commutates current from the power source through the primary of a step-up transformer, the transformer preferably being a Tesla-type coil potted in silicone elastomer to suppress corona discharge and mechanically coupled to the printed-circuit board by a compliant standoff. The transformer's secondary delivers high-potential output pulses through a medical-grade, double-insulated high-tension cable that is routed through a strain-relieved bulkhead fitting before attachment to a laminated electrode assembly.

In some aspects, the laminated electrode assembly is produced by bonding a planar copper sheet having a thickness between 0.5 mm and 1.0 mm and a surface area of at least 645 cm² to a polymethyl-methacrylate dielectric plate at least 5 mm thick, the bonding achieved with a UV-curable, low-shrink epoxy after the dielectric plate has been laser-cut or CNC-machined to include 45-degree beveled peripheral edges. When the copper electrode is driven by the high-potential pulses, the dielectric layer capacitively delivers a pulsed electromagnetic field to tissue positioned proximate the plate, the short pulse width and low duty cycle producing a shallow penetration depth that stimulates muscular response, enhances cellular metabolism, supports collagen formation, reduces concentration of free radicals, and promotes endorphin release in the living subject. A resistive discharge path of at least one megohm is connected between a practitioner-contact surface—such as a conductive-polymer grounding mat—and earth ground so that displacement current generated by the PEMF is dissipated safely while enabling a therapist-guided modality in which a practitioner who is standing on the grounding interface can, through direct skin contact and massaging motions, locally concentrate the PEMF at any region selected on the subject's body.

All high-tension wiring is preferably confined to an EMI-shielded aluminum chassis section that is powder-coated before electronic sub-assemblies are installed, the wiring schedule observing at least 5 kV of creepage and clearance distance between energized conductors and user-accessible surfaces. The printed-circuit board inside that compartment carries the pulse-rate generator section, the MOSFET switching section, the capacitor bank, a removable power-supply module, and a microprocessor that is factory-programmed to prevent duty-cycle values above 30% whenever the repetition rate exceeds 20 Hz. Non-volatile memory automatically stores session parameters such as start time, stop time, selectable repetition rate, and output-pulse amplitude, and a wireless communications module can transmit the stored data to an external computing device while also receiving firmware updates that alter pulse-width-modulation profiles or that invoke an adaptive control algorithm which sweeps the selectable repetition rate through a predetermined spectrum of frequencies identified as beneficial for neuromuscular stimulation. These beneficial, predetermined frequencies and/or spectrum(s) preferably may be identified by a look-up table. A capacitive-sense touchscreen positioned on the enclosure may provide manual frequency selection within the aforementioned 6 Hz-to-36 Hz window and display real-time numerical indications of output voltage and frequency.

During manufacture, the copper-and-dielectric laminate is preferably prepared first, the Tesla coil is potted, the high-tension cable is dressed through its fitting, and the main board is mounted in the shielded compartment. After mechanical coupling of the coil to the board, the enclosure is closed, and the complete apparatus is calibrated by measuring electromagnetic field strength at the dielectric surface while adjusting pulse-width parameters until the desired field is obtained. Session calibration is followed by installation of the system, together with its laminated electrode, the grounding mat, and optional accessories such as massage cream to reduce skin friction, in a portable transport case sized for airline carry-on use and fitted with foam compartments that protect each component. The finished system therefore comprises a pulse-gated PEMF generator, the laminated electrode, the resistive grounding interface, data-logging and wireless subsystems, and thermal-management features; it is capable of battery-operated field use without interrupting firmware-controlled data logging.

In some aspects, enclosure 102 may be machined from 2.0-millimeter-thick 5052-H32 aluminum sheet that is subsequently bent along radiused brake lines to ±0.25 millimeter tolerance, TIG-welded at seam corners, and powder-coated with a polyester-epoxy hybrid resin to a nominal thickness of 75 microns. The interior surfaces may be pre-treated with a conversion-coating process to improve corrosion resistance and electromagnetic shielding effectiveness. A continuous thermoplastic elastomer gasket may be seated within a 2.2-millimeter wide rabbet formed along the clamshell mating rim, such that when over-center draw latches close the lid, an IP-54 splash barrier may be established without compressing board-level components.

In certain aspects, an internal sub-chassis may be fabricated from 1.6-millimeter anodized aluminum and standoff-mounted to the enclosure floor via M4 stainless machine screws torqued to 1.2 newton-meters, thereby establishing a rigid, low-impedance ground plane beneath pulse-rate generator 104 and input drive FET switch 106. Four 6.4-millimeter diameter threaded PEM® nuts may be pressed into the chassis corners to receive shock-mount grommets that isolate ignition coil 112, limiting vibrational energy transmission above 10 g RMS and extending solder-joint fatigue life. A 0.05-millimeter copper-foil shield may be laminated between the sub-chassis and a FR-4 insulating sheet to attenuate radiated fields above 30 megahertz.

In various aspects, ignition coil 112 may be over-mold potted with addition-cure silicone having a dielectric breakdown strength greater than 20 kilovolts per millimeter, thereby sealing the high-turn secondary and preventing partial-discharge events in humid environments. A pair of molded polypropylene bushings may center the coil's ferrule within a 51-millimeter aperture, while a silicone-gel strain relief may cradle primary-lead exit points to minimize conductor work-hardening during transport.

In other aspects, copper plate 502B may be fabricated from C110 electrolytic-tough-pitch copper exhibiting a minimum tensile strength of 200 megapascals. The plate may be face-milled to Ra≤0.8 micron before adhesive application to ensure intimate bond-line wetting with the PMMA dielectric panel. Laser-cut 45-degree edge chamfers may remove burrs and generate a controlled perimeter angle that reduces electric-field edge enhancement. The acrylic cover may be laser-cut from 5-millimeter cast PMMA, the cut path programmed to ±0.05 millimeter precision, then solvent-polished for optical clarity that facilitates QC inspection of sub-surface bonds.

In some implementations, final assembly may proceed by first anchoring pulse-storage capacitors 114A and 114B through nylon shoulder washers that insulate the canisters from chassis potential while maintaining 10-millimeter spacing between adjacent capacitor shells to mitigate dielectric heating. The capacitor tie-bar may be copper bus-plate drilled with 6.3-millimeter clearance holes and fastened via M5 low-profile screws tightened to 2.8 newton-meters; torque values may be verified with a click-type wrench to limit thread galling. Silicone conformal coating may then be selectively applied around soldered joints to resist vibration and moisture ingress.

In several aspects, a modular variant may substitute a split-plate electrode assembly in which two discrete copper regions 512A and 512B are independently energizable. Blade-style jumpers accessible through a captive-screw window may enable series, parallel, or isolated addressing by the pulse-rate generator, thereby allowing bilateral treatment protocols without repositioning the patient. Alternatively, a hinged dielectric frame may permit stackable spacers to vary plate-to-body distance, which may tune coupling capacitance for pediatric versus adult patients.

In many aspects, frequency adjustment knob 902 and amplitude knob 904 may incorporate detent-equipped potentiometer shafts that engage with polyurethane ratchet wheels molded at 36-position intervals, providing tactile feedback approximately every 1 hertz for frequency and every 0.3 volt for amplitude. A non-contact Hall-effect encoder may replace the analog potentiometer in a digital-control configuration, enabling pulse-width modulation values to be stored in non-volatile memory 1502 for repeatable clinical protocols.

In certain aspects, thermal management may be enhanced by coupling input drive FET switch 106 to an extruded aluminum heat sink featuring 15 fins each 1.2 millimeter thick and spaced 4.0 millimeters apart to optimize convection at fan velocities of 1.5 meters per second. The sink may be bonded with a graphite-based phase-change interface pad that exhibits thermal impedance below 0.2° C.-cm²/Watt. A 40×40-millimeter axial fan may draw air through a stainless-steel EMI mesh filter and exhaust through rear vent louvers angled downward at 45 degrees to prevent liquid ingress.

For instance, safety interlock wiring may route through reed switches embedded in both lid hinges; opening the lid may de-energize gate drive within 10 milliseconds. In addition, a 15-kilovolt gas-discharge tube may be placed across connector 602 to clamp any residual energy upstream of the dielectric plate during fault conditions. Surge arrestors may be deliberately positioned at nodes exhibiting low source impedance, and MOVs may be connected line-to-neutral at inlet 906 to absorb lightning-induced transients.

In some aspects, calibration may involve coupling a handheld Gauss-meter probe 1804 two centimeters above the center of plate 502B while executing an automated four-step sweep routine that incrementally raises duty cycle until magnetic flux density registers between 0.5 and 3.0 gauss. Firmware may then compute an adjustment factor stored in EEPROM that scales subsequent duty-cycle commands, ensuring consistent field strength across manufacturing lots. A recessed trimmer potentiometer accessible via sealable grommet may fine-tune gate-bias voltage to maintain a 5-ampere peak primary-current target.

In other aspects, the apparatus may be deployed in veterinary physiology laboratories where hoof or paw placement upon a reduced-size dielectric pad may aid ligament recovery. The same core architecture may be packaged in a rack-mount chassis for aerospace-medicine research, where electrodes may line crew-recovery couches. Industrial facilities may integrate the system into ergonomic stations to reduce musculoskeletal strain among repetitive-task workers.

In several aspects, transport durability may be addressed by molding corner bumpers of thermoplastic polyurethane, hardness Shore A 85, which may over-mold directly onto the case corners via two-shot injection. Stainless-steel latch arms may pivot on 3-millimeter dowel pins retained by e-clips rated to 9 kN shear, preventing latch disengagement during 1-meter drop testing to MIL-STD-810G, method 516.6.

In many aspects, functional redundancy may be achieved by paralleling two MOSFET devices 106A and 106B with individual gate resistors; failure of one device may trigger an on-die temperature sensor to signal microcontroller 1402, which, in turn, may reduce duty cycle to 10 percent and illuminate fault LED 1420. A secondary path consisting of quench resistor 1432 and bleed diode 1434 may discharge residual capacitor energy within 200 milliseconds should gate drive abruptly cease.

For example, periodic maintenance may include unscrewing connector 602's quarter-turn collar, exposing a bayonet-style contact rated to 25,000 volt-impulse. Users may wipe the dielectric surface with isopropyl alcohol and inspect for crazing; if surface roughness exceeds 3 microns Ra, technicians may polish with a micro-mesh abrasive pad and reapply an anti-static silicone wipe to restore uniform field distribution. Firmware may log maintenance cycles via a checksum in session-data block 1608, enabling compliance audits.

In some embodiments, future production lines may adopt an over-center lever clamp that secures electrode assemblies to examination tables, allowing quick height adjustments without tools. Optional pneumatic cylinders may raise or tilt the plate to accommodate bariatric patients, while positional feedback sensors may relay plate inclination to the control unit, where stored lookup tables may automatically derate pulse amplitude to maintain safe displacement current density.

In various aspects, the disclosed structural details may focus on heat-sink fin geometry, electrode encapsulation thickness, modular cable connectors, and chassis reinforcement ribs. The breadth of materials and assembly modalities presented herein may enable the technology to be tailored to marine, austere military, or field-research environments, thereby underscoring industrial applicability across a wide spectrum of therapeutic and wellness platforms.

The foregoing descriptions are presented solely as exemplary implementations that may be combined, omitted, or varied, and should not be interpreted to restrict the scope of the appended claims.

### CONCLUSION

For clarity of explanation, the above description has focused on a representative sample of all possible embodiments, a sample that teaches the principles of the invention and conveys the best mode contemplated for carrying it out. The invention is not limited to the described embodiments. Well known features may not have been described in detail to avoid unnecessarily obscuring the principles relevant to the claimed invention. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed alternatives, variations, modifications, and equivalents are within the literal scope of the following claims, and others are equivalent. The claims may be practiced without some or all of the specific details described in the specification. In many cases, method steps described in this specification can be performed in different orders than that presented in this specification, or in parallel rather than sequentially. It is to be understood that the present technology contemplates that, to the extent possible, one or more features of any implementation can be combined with one or more features of any other implementation.

While the above description details certain embodiments of the invention and describes the best mode contemplated, no matter how detailed the above appears in text, the invention can be practiced in many ways. Therefore, implementation details may vary considerably while still being encompassed by the invention disclosed herein. Particular terminology used when describing certain features or aspects of the invention should not be taken to imply that the terminology is being redefined herein to be restricted to any specific characteristics, features, or aspects of the invention with which that terminology is associated.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Each of these non-limiting examples may stand on its own or may be combined in various permutations or combinations with one or more of the other examples.

What is claimed is:

1. A pulsed electromagnetic field (PEMF) therapy apparatus comprising:
a power source;
a pulse-rate generator configured to produce gating pulses;
a solid-state switching stage including at least one Metal Oxide Semiconductor Field Effect Transistor (MOSFET) having a control terminal driven by the gating pulses and a conduction path arranged to periodically switch current from the power source;
a step-up transformer whose primary winding is coupled to the solid-state switching stage and whose secondary winding provides high-potential output pulses directed via high-tension insulated cable to a dielectrically encased copper plate electrode;
a copper electrode; and
a dielectric layer positioned over the copper electrode, the copper electrode being coupled to the high-potential output so that the dielectric layer capacitively delivers the pulses as a pulsed electromagnetic field (PEMF) to a body region positioned proximate the dielectric layer, wherein the PEMF is configured to stimulate cellular metabolism.

2. The apparatus of claim 1, wherein the power source is a direct-current supply delivering between 3 volts and 14 volts and further comprising an energy-storage capacitor bank electrically coupled to the power source.

3. The apparatus of claim 1, wherein the pulse-rate generator comprises dual 555-type timer integrated circuits or a microprocessor separately adjustable for frequency and pulse width, a selectable repetition rate being within 6 hertz to 36 hertz.

4. The apparatus of claim 1, wherein the copper electrode is a planar conductor having a thickness between 0.5 millimeter and 1.0 millimeter and an area of at least 645 square centimeters, configured to enhance cellular health by supporting collagen formation and reducing free radicals.

5. The apparatus of claim 1, further comprising a resistive discharge path of at least 1.0 megohm connected between a practitioner-contact surface and earth ground to dissipate displacement current generated by the PEMF.

6. The apparatus of claim 1, further comprising an enclosure including thermal-management components selected from a group consisting of a heat sink bonded to the MOSFET, a fan, and a temperature sensor configured to disable the pulse-rate generator upon detecting an over-temperature condition.

7. The apparatus of claim 1, wherein the dielectric layer comprises a polymethyl methacrylate sheet of at least 5 millimeters thickness adhesively bonded to the copper electrode with epoxy.

8. The apparatus of claim 1, wherein the high-potential output pulses are configured with a short pulse width and low duty cycle to produce a shallow penetration depth, stimulating muscular response without deep tissue exposure.

9. A method of manufacturing a PEMF therapy apparatus, the method comprising:
providing a laminated electrode assembly by bonding a copper conductive sheet to a dielectric plate;
potting a step-up Tesla coil within silicone elastomer to suppress corona discharge;
mounting a printed-circuit board containing a pulse-rate generator section and a MOSFET-based switching section inside a shielded compartment of an apparatus enclosure;
mechanically coupling the step-up Tesla coil to the printed-circuit board through a compliant standoff; and
electrically interconnecting the printed-circuit board, the step-up Tesla coil, the laminated electrode assembly, and a removable power-supply module in accordance with a wiring schedule.

10. The method of claim 9, further comprising routing a medical-grade, double-insulated high-tension cable through a strain-relieved bulkhead fitting before attachment to the laminated electrode assembly and using a UV-curable low-shrinkage adhesive when bonding the copper conductive sheet to the dielectric plate.

11. The method of claim 9, further comprising laser-cutting or CNC machining the dielectric plate to include beveled peripheral edges having a 45-degree chamfer, thereby reducing edge sharpness.

12. The method of claim 9, further comprising programming a microprocessor on the printed-circuit board with firmware that limits a duty cycle of pulses to less than 30 percent when a selectable repetition rate exceeds 20 hertz.

13. The method of claim 9, wherein the shielded compartment comprises an aluminum chassis section that is powder-coated prior to installation of electronic sub-assemblies, the wiring schedule maintaining at least 5 kilovolts of creepage and clearance distance between high-tension conductors and user-accessible surfaces.

14. A pulsed PEMF therapy system comprising:
a pulse-gated PEMF generator including a power source, a pulse-rate generator, a MOSFET-based switching stage coupled to the power source and driven by the pulse-rate generator, a step-up transformer providing high-potential output pulses at a high-potential output, a copper electrode, and a dielectric layer positioned over the copper electrode and coupled to the high-potential output so that the dielectric layer capacitively delivers the high-potential output pulses as a PEMF into an adjacent region proximate the dielectric layer; and
a resistive grounding interface, wherein:
the system provides for a therapy modality, wherein when an operator is in conductive contact with the resistive grounding interface and simultaneously contacts a target tissue, displacement current flows through the operator and locally concentrates the PEMF at a region of a living subject selected by the operator.

15. The system of claim 14, further comprising a portable transport case dimensioned for carry-on use and having foam compartments configured to support the pulse-gated PEMF generator, the copper electrode, and the resistive grounding interface, the resistive grounding interface comprising a mat formed of conductive-polymer material incorporating a resistance of at least one megohm between the mat and earth ground.

16. The system of claim 14, wherein the pulse-rate generator is manually adjustable within 6 hertz to 36 hertz and is operatively coupled to a capacitive-sense touchscreen that displays numerical indications of output voltage and frequency.

17. The system of claim 14, wherein the MOSFET-based switching stage is rated for at least 600 volts drain-to-source and is mounted on a graphite-based thermal pad to a thermal heatsink, whose constituent components are housed inside an EMI-shielded compartment of an enclosure that houses the pulse-gated PEMF generator.

18. The system of claim 14, further comprising non-volatile memory configured to automatically store session parameters including start time, stop time, selectable repetition rate, and output-pulse amplitude for each of multiple therapy sessions.

19. The system of claim 14, wherein the pulse-rate generator further comprises an adaptive control algorithm that sweeps a selectable repetition rate through a predetermined spectrum of frequencies identified by a look-up table as beneficial for neuromuscular stimulation.

20. The system of claim 14, further comprising a massage cream applied to skin of the living subject to reduce friction during practitioner-guided application of the PEMF.
